(19) United States
(12) Patent Application Publication
(10) Pub. No.: US 2025/0336311 A1
(43) Pub. Date: Oct. 30, 2025

(54) ENDOSCOPIC SURGERY TRAINING DEVICE

(71) Applicant: EndoRobotics Co., Ltd., Seoul (KR)

(72) Inventors: Byung Doo Min, Seoul (KR); Yong Tae Seo, Seoul (KR); Ye Chan Seo, Seoul (KR); Erkhes Bilegt, Seoul (KR); Yong Hyun Hwang, Seoul (KR); Jeong Han Kim, Seoul (KR); Soo Yong Choi, Seoul (KR); Kyung Nam Kim, Seoul (KR); Byung Gon Kim, Seoul (KR)

(21) Appl. No.: 19/193,190

(22) Filed: Apr. 29, 2025

(30) Foreign Application Priority Data
Apr. 30, 2024 (KR) 10-2024-0057621
May 14, 2024 (KR) 10-2024-0063057
Apr. 21, 2025 (KR) 10-2025-0051715

(51) Int. Cl. G09B 23/28

(52) U.S. Cl. CPC G09B 23/285

(57) ABSTRACT

An endoscopic surgery training apparatus is disclosed. The endoscopic surgery training apparatus according to an embodiment of the present disclosure may include a housing including a training space formed inside for training a surgery; a holder configured to fix a training target member; a driving part including a holder support member to which the holder is coupled, and configured to change a position of the holder coupled to the holder support member within the training space through at least one degree-of-freedom movement so as to change a training target member fixed to the holder to a specific simulation position of an organ for training within the training space; a channel forming part formed in the housing so as to form an entry path of an endoscopic device entering the training space from the outside; and a controller for controlling the driving part.

### CROSS-REFERENCE TO RELATED APPLICATION(S)

This application claims priority to and the benefit of Korean Patent Application No. 10-2024-0057621, filed on Apr. 30, 2024, Korean Patent Application No. 10-2024-0063057, filed on May 14, 2024, and Korean Patent Application No. 10-2025-0051715, filed on Apr. 21, 2025, the disclosures of which are incorporated herein by reference in their entirety.

### BACKGROUND

### Field

The present disclosure relates to an endoscopic surgery training apparatus.

### Discussion of Related Technology

An endoscopic apparatus is an apparatus that is designed to insert a machine into the body to check for lesions in an organ.

Since endoscopic surgery or procedures using such an endoscopic apparatus are performed in a narrow space inside the human body, a lot of training and experience may be required to increase skill.

### SUMMARY

Surgical training apparatuses for training endoscopic surgery may be provided. However, in such surgical training apparatuses may only provide a model that simulates parts of the body into which the endoscopic apparatus is inserted, and may only train the act of observing and examining lesions inside the body, and would not be able to provide sufficient training for surgery.

The present disclosure is to provide an endoscopic surgery training apparatus that can implement a specific part of a target organ similar to an actual organ.

According to an aspect of the present disclosure, provided is an endoscopic surgery training apparatus, including a housing including a training space formed inside for training a surgery, and a support plate forming a bottom surface of the training space; a holder configured to fix a training target member; a driving part including a holder support member to which the holder is coupled, and configured to change a position of the holder coupled to the holder support member within the training space through at least one degree-of-freedom movement so as to change the training target member fixed to the holder to a specific simulation position of an organ for training within the training space; a channel forming part formed in the housing so as to form an entry path of an endoscopic device entering the training space from the outside; and a controller for controlling the driving part.

In this case, the driving part may change the position of the holder coupled to the holder support member within the training space through a four-degree-of-freedom movement including a first linear movement, a first rotation, a second rotation and a second linear movement.

In this case, the driving part may include a first linear driving part configured to linearly move the holder support member along the X-axis direction; a first rotational driving part configured to rotate the holder support member around a first rotation axis; a second rotational driving part configured to rotate the holder support member around a second rotation axis; and a second linear driving part configured to linearly move the holder support member in a direction perpendicular to the second rotation axis, and in the holder support member, a first part to which the holder is coupled may be arranged so as to be parallel to the second rotation axis and to be spaced apart from the second rotation axis by a certain distance.

In this case, when the holder support member is placed in an initial position by the driving part while the holder is coupled to the first part, the center of the holder may be placed in a straight line with the first rotation axis, which is the central axis for a first rotation of the holder support member.

Meanwhile, the holder may include a holder body including a mounting part that is detachably coupled to one side of the driving part and a plurality of leg parts that extend from the mounting part, and a plurality of gripping members that are detachably coupled to the plurality of leg parts so as to be able to grip the training target member.

In this case, the holder may include a plurality of first magnetic members that are provided on each of the plurality of leg parts, and a plurality of second magnetic members that are provided on each of the plurality of gripping members so as to correspond to the plurality of first magnetic members, and each of the plurality of gripping members may be detachably coupled to the holder body through the first magnet member and second magnet member corresponding to each other.

Meanwhile, the driving part may include a first linear driving part including a moving member that reciprocates linearly along the X-axis with respect to the support plate via a first driving motor; a first rotational driving part including a second driving motor that is fixedly coupled to the moving member, a first rotation axis that rotates around the Z-axis that is perpendicular to the X-axis with respect to the moving member through a driving force of the second driving motor, and a first mounting member that is fixedly coupled to the first rotation axis; a second rotational driving part including a third driving motor that is fixedly coupled to the first mounting member, a second rotation axis that rotates around an axis that is parallel to the XY plane and perpendicular to the Z axis with respect to the first mounting member through a driving force of the third driving motor, and a second mounting member that is fixedly coupled to the second rotation axis; and a second linear driving part including a fourth driving motor that is fixedly coupled to the second mounting member, and provides a driving force for reciprocating a linear movement of the holder support member along a direction perpendicular to an axis that is parallel to the XY plane and perpendicular to the Z-axis with respect to the second mounting member.

In this case, the endoscopic surgery training apparatus may further include a third magnet member provided on the holder support member, and a fourth magnet member provided on the holder to correspond to the third magnet member, and the holder may be detachably coupled to the holder support member via the third magnetic member and the fourth magnetic member.

Meanwhile, the first linear driving part may further include a first guide rail fixed to the support plate; a first slider movably coupled to the first guide rail so as to be able to move linearly in the X-axis direction along the first guide rail; a first rack gear fixedly coupled to one side of the moving member so as to be parallel to the longitudinal direction of the first guide rail; and a first pinion gear meshed with the first rack gear while being axially coupled to the first driving motor so as to be able to rotate by the first driving motor, and the moving member may move in a reciprocating linear manner along the X-axis with respect to the support plate through the first slider that is fixed to one side.

Meanwhile, the first rotational driving part may further include a first driving gear that is axially coupled to the second driving motor, and a first driven gear that is axially coupled to the first rotation axis and meshes with the first drive gear, and the first mounting member may rotate around the Z-axis, which is perpendicular to the X-axis, with respect to the moving member through a rotation of the first rotation axis.

Meanwhile, the second rotational driving part may further include a second driving gear that is axially coupled to the third driving motor, and a second driven gear that is axially coupled to the second rotation axis and meshes with the second driving gear, and the second mounting member may rotate around an axis perpendicular to the Z-axis with respect to the first mounting member through a rotation of the second rotation axis.

Meanwhile, the second linear driving part may further include a second guide rail fixed to the second mounting member; a second slider movably coupled to the second guide rail so as to be able to reciprocally move linearly along the second guide rail; a second rack gear fixedly coupled to one side of the holder support member so as to be parallel to the longitudinal direction of the second guide rail; and a second pinion gear meshed with the second rack gear while being axially coupled to the fourth driving motor so as to be able to rotate by the fourth driving motor, and the holder support member may move in a reciprocating linear manner along the second guide rail with respect to the second mounting member through the second slider that is fixed to one side.

Meanwhile, the controller may drive the driving part such that the holder support member moves while the training target member is changed to a specific simulation position of an organ for training by driving the driving part.

Meanwhile, the channel forming part may include a channel case coupled to the housing; a path part formed to be recessed from one surface of the channel case so as to form an entry path for the endoscopic device; and a path maintenance member formed in a hollow shape such that the endoscopic device passes through and detachably coupled to the path part, wherein the path part may include a first path forming part formed to be connected to the training space and have a certain length; and a second path forming part and a third path forming part each extending from the first path forming part so as to branch in different directions from the other end of the first path forming part, and wherein the path maintenance member may be mounted on the path part so as to be positioned at the first path forming part and the second path forming part to form a first entry path, or may be mounted on the path part so as to be positioned at the first path forming part and the third path forming part to form a second entry path.

In this case, the path maintenance member may include a hollow connecting pipe having a predetermined length, a first coupling port provided at one end of the connecting pipe so as to be detachably coupled to one end of the first path forming part, and a second coupling port provided at the other end of the connecting pipe so as to be detachably coupled to one end of the second path forming part or one end of the third path forming part.

Meanwhile, while the holder is changed to a specific simulation position of an organ through the driving part, the training target member that is mounted on the holder may implement an inner wall of the organ, and an endoscopic device that enters the training space through the channel forming part may be positioned such that a tip faces one surface of the training target member.

Meanwhile, the endoscopic surgery training apparatus may further include an auxiliary support part positioned at a bottom of the channel forming part so as to support a portion of the endoscopic device that has entered the training space through the channel forming part.

In this case, the auxiliary support part may include an auxiliary support configured to support a portion of an endoscopic device that has entered the training space, and an auxiliary driving part configured to change a position of the auxiliary support through a fifth driving motor within the training space, and the controller may drive the auxiliary driving part to move the auxiliary support toward the training target member when the training target member changes to a preset specific simulation position.

Meanwhile, the auxiliary driving part may further include a third slider fixed to a coupling member that is coupled to the support plate; a third guide rail that is movably coupled to the third slider so as to be able to move linearly in the X-axis direction and has the auxiliary support fixedly coupled to one side; a third rack gear formed along the X-axis direction on one side of the third guide rail; and a third pinion gear that is axially coupled to the fifth driving motor so as to be able to rotate by the fifth driving motor and meshes with the third rack gear, and the auxiliary support may move toward the training target member through a linear movement of the third guide rail.

Meanwhile, the endoscopic surgery training apparatus may further include an operating part for user operation, wherein the operating part may generate a user input signal to change the training target member to a specific simulation position of an organ.

Meanwhile, the endoscopic surgery training apparatus may further include a connector provided on one side of the housing so as to be electrically connected to the holder support member, and a portion of which is exposed to the outside of the housing and is coupled to a terminal of a cable that is electrically connected to a cautery, and the holder that is coupled to the holder support member may be electrically connected to the connector.

According to the above configuration, the endoscopic surgery training apparatus according to the present disclosure can implement a specific part of an organ that is targeted by a training target member similarly to an actual organ. Through this, the user can perform surgical training in an environment similar to an actual environment, thereby increasing the skill level

The effects of the present disclosure are not limited to the above effects, and should be understood to include all effects that can be inferred from the configuration of the invention described in the detailed description or claims of the present disclosure.

### DESCRIPTION OF THE DRAWINGS

FIG. 1 is a drawing showing an endoscopic surgery training apparatus according to an embodiment of the present disclosure.

FIG. 2 is a drawing showing a state in which a door is opened and a channel case is withdrawn in FIG. 1.

FIG. 3 is a drawing schematically showing the interior of FIG. 1.

FIG. 4 is a drawing schematically showing the arrangement relationship of a driving part, an auxiliary driving part and a channel forming part arranged inside the housing in FIG. 1.

FIG. 5 is a block diagram showing the main components controlled by a controller in the endoscopic surgery training apparatus according to an embodiment of the present disclosure.

FIG. 6 is a drawing showing a holder that can be applied to the endoscopic surgery training apparatus according to an embodiment of the present disclosure.

FIG. 7 is a drawing showing a state in which a holder and a holder support member are separated in the endoscopic surgery training apparatus according to an embodiment of the present disclosure.

FIG. 8 is a drawing showing a support plate, a driving part and an auxiliary driving part extracted from the endoscopic surgery training apparatus according to an embodiment of the present disclosure.

FIG. 9 is a drawing of a driving part and a holder extracted from the endoscopic surgical training apparatus according to an embodiment of the present disclosure, with some of the components separated.

FIG. 10 is a drawing showing a lower part of a moving member in the endoscopic surgery training apparatus according to an embodiment of the present disclosure.

FIG. 11 is a drawing showing the coupling relationship of a first rotational driving part in the endoscopic surgery training apparatus according to an embodiment of the present disclosure.

FIG. 12 is a drawing showing the coupling relationship of a second rotational driving part in the endoscopic surgery training apparatus according to an embodiment of the present disclosure.

FIG. 13 is a drawing showing FIG. 12 from another direction, showing a state in which the second mounting member is removed.

FIG. 14 is a drawing showing the coupling relationship of a second linear driving part in the endoscopic surgery training apparatus according to an embodiment of the present disclosure.

FIG. 15 is a drawing showing a state in which a holder support member moves linearly through a second linear driving part in the endoscopic surgery training apparatus according to an embodiment of the present disclosure.

FIG. 16 is a drawing showing a channel forming part that is applicable to the endoscopic surgery training apparatus according to an embodiment of the present disclosure, showing a state in which a path maintenance member is mounted to be positioned at the first path forming part and the second path forming part.

FIG. 17 is a drawing showing a channel forming part that is applicable to the endoscopic surgery training apparatus according to an embodiment of the present disclosure, showing a state in which a path maintenance member is mounted to be positioned at the first path forming part and the third path forming part.

FIG. 18 is a drawing showing an auxiliary driving part that is applicable to the endoscopic surgery training apparatus according to an embodiment of the present disclosure.

FIG. 19 is a drawing looking at FIG. 18 from another direction.

FIG. 20 is a drawing showing a state in which the endoscopic surgery training apparatus according to an embodiment of the present disclosure is in use, showing a state in which a holder and a training target member are disposed to be positioned at different specific positions.

FIG. 21 is a drawing showing a state in which the endoscopic surgery training apparatus according to an embodiment of the present disclosure is in use, showing a state in which a holder and a training target member are disposed to be positioned at different specific positions.

FIG. 22 is a diagram showing a state in which the endoscopic surgery training apparatus according to an embodiment of the present disclosure is in use, showing a state in which a holder and a training target member are disposed to be positioned at other specific positions.

### DETAILED DESCRIPTION OF EMBODIMENTS

Hereinafter, with reference to the attached drawings, embodiments of the present disclosure will be described in detail so that those skilled in the art can easily practice the present disclosure. The present disclosure may be implemented in various different forms and is not limited to the embodiments described herein. In order to clearly explain the present disclosure, parts that are not related to the description in the drawings have been omitted, and the same reference numerals have been assigned to the same or similar components throughout the specification.

The words and terms used in the present specification and claims should not be interpreted as having limited meanings in their usual or dictionary sense, but should be interpreted as having meanings and concepts that conform to the technical idea of the present disclosure according to the principle that the inventor can define terms and concepts in order to explain embodiments of his or her invention in the best way.

In addition, the X-axis, Y-axis and Z-axis used in the present specification and claims may be three axes that are orthogonal to each other, and the axis perpendicular to the Z-axis may be any straight line that is arranged perpendicular to the Z-axis on the XY plane.

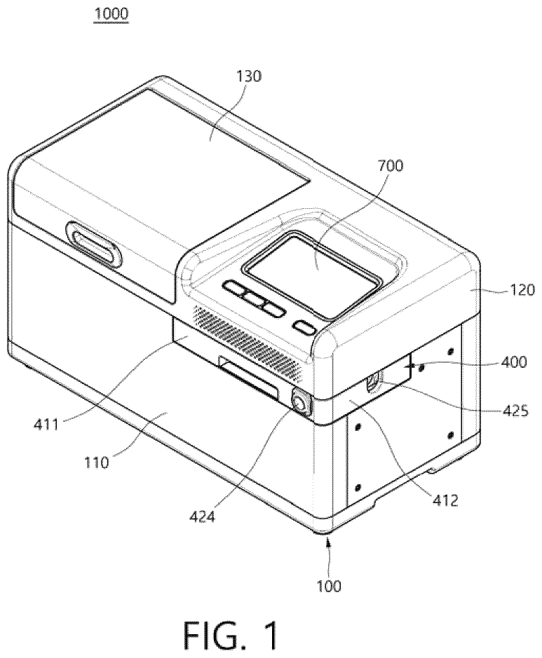

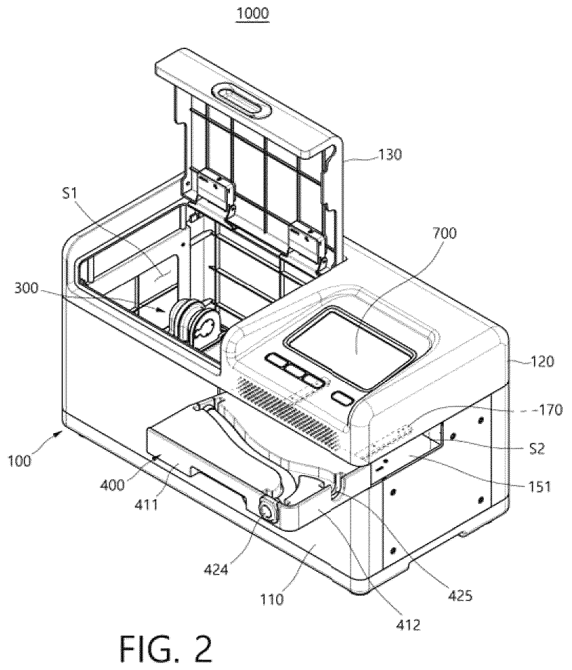

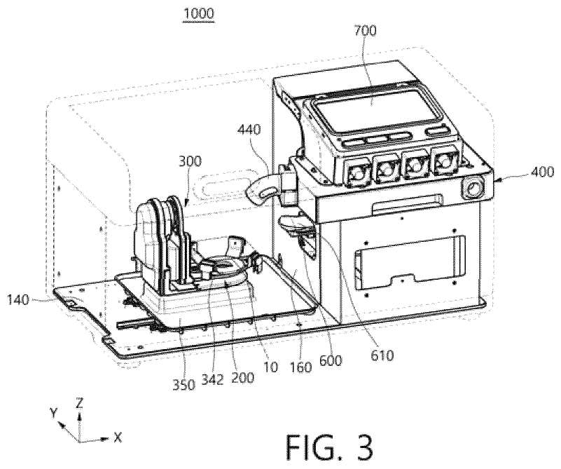

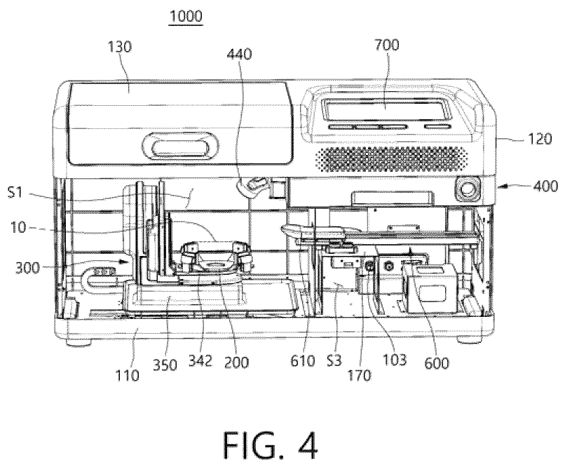

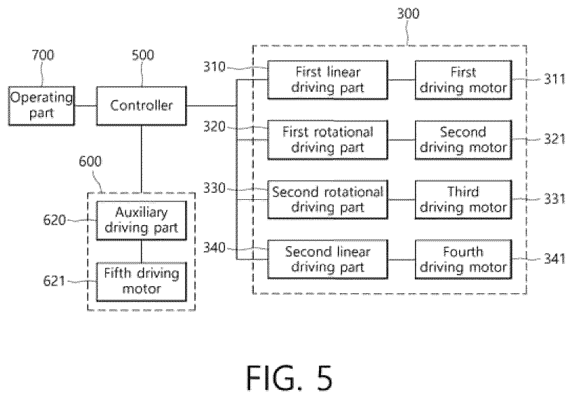

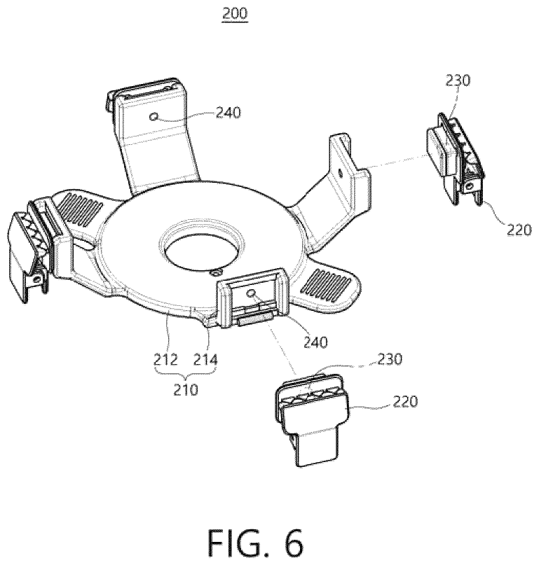

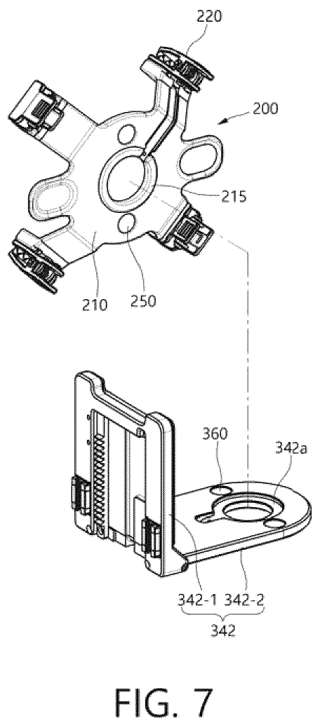

FIG. 1 is a drawing showing an endoscopic surgery training apparatus according to an embodiment of the present disclosure, FIG. 2 is a drawing showing a state in which a door is opened and a channel case is withdrawn in FIG. 1, FIG. 3 is a drawing schematically showing the interior of FIG. 1, FIG. 4 is a drawing schematically showing the arrangement relationship of a driving part, an auxiliary driving part and a channel forming part arranged inside the housing in FIG. 1, FIG. 5 is a block diagram showing the main components controlled by a controller in the endoscopic surgery training apparatus according to an embodiment of the present disclosure, FIG. 6 is a drawing showing a holder that can be applied to the endoscopic surgery training apparatus according to an embodiment of the present disclosure, and FIG. 7 is a drawing showing a state in which a holder and a holder support member are separated in the endoscopic surgery training apparatus according to an embodiment of the present disclosure.

Referring to FIGS. 1 to 7, the endoscopic surgery training apparatus 1000 according to an embodiment of the present disclosure may improve the surgical skill of a user, such as a doctor, by implementing an environment similar to an actual surgery.

That is, the endoscopic surgery training apparatus 1000 according to an embodiment of the present disclosure may implement a specific part of an organ to improve surgical skill by changing a training target member 10 for simulating an organ such as the stomach or the large intestine to a specific position.

For example, the endoscopic surgery training apparatus 1000 according to an embodiment of the present disclosure may implement a specific part of the stomach or the large intestine by changing the position of the training target member 10, thereby improving the skill of endoscopic surgery for a specific part of the stomach or the large intestine.

The term ‘training target member’ may be replaced with the term ‘training target model’.

The training target member 10 may be formed such that one surface facing the tip of an endoscopic device 20 is formed by a combination of a curved surface and a flat surface, or is formed only by a curved surface, or is formed only by a flat surface.

In the present disclosure, the training target member 10 may be fixed through a holder 200 described below, and one surface of the training target member 10 facing the tip of the endoscope device 20 in a state where the training target member 10 is changed to a specific position may implement the inner wall of an organ such as the stomach or large intestine.

Herein, the endoscope device 20 may include not only an endoscope device including a camera and a lens module to observe lesions inside the body, but also a surgical tool such as surgical forceps, surgical scissors, a surgical knife, a surgical robot arm that can grasp body tissue or perform a rotational movement and the like that are used in endoscopic surgery or procedures together with the endoscope device.

Accordingly, the user may change the position of the holder 200 to which the training target member 10 is fixed such that the training target member 10 implements a specific part of the target organ, thereby increasing surgical proficiency for a specific part of the organ that requires training.

In addition, the endoscopic surgery training apparatus 1000 according to an embodiment of the present disclosure is implemented in a portable size such that it can be used in various places without being restricted by location.

To this end, the endoscopic surgery training apparatus 1000 according to an embodiment of the present disclosure may include a housing 100, a holder 200, a driving part 300, a channel forming part 400 and a controller 500 as shown in FIGS. 1 to 5.

The housing 100 may form an overall outer shape. Such a housing 100 may include a training space (S1) formed inside.

In such a case, the holder 200 and the driving part 300 may be placed in a training space (S1), and the training target member 10 may be fixed to the holder 200.

In addition, the endoscope device 20 may enter the training space (S1) through the channel forming part 400.

Accordingly, the user may perform training on a specific part of an organ requiring training by changing the position of the training target member 10 fixed to the holder 200 through the driving part 300 within the training space (S1).

In this case, the housing 100 may include a door 130 formed in an area corresponding to the training space (S1).

For example, the housing 100 may include a first housing 110 in the shape of a body in which the training space (S1) is formed, a second housing 120 coupled to the first housing 110 so as to cover the training space (S1), and a door 130 openably coupled to the second housing 120 so as to be positioned above the training space (S1).

Accordingly, as shown in FIG. 2, a user may open and close the door 130 to open the training space (S1).

The holder 200 may fix the training target member 10, and may be coupled to one side of the driving part 300.

For example, the holder 200 may be coupled to a holder support member 342 of the driving part 300.

Herein, the holder support member 342 may include a second part 342-1 to which a second slider 344 and a second rack gear 345 described below are fixed, as shown in FIG. 7, and a first part 342-2 to which the holder 200 is detachably coupled.

In this case, as shown in FIG. 6, the holder 200 may include a holder body 210 detachably coupled to the first part 342-2, and a plurality of gripping members 220 coupled to the holder body 210 so as to be able to grip the training target member 10.

In addition, the plurality of gripping members 220 may be arranged on one side of the holder body 210 so as to be spaced apart from each other, and may each grip a portion of the training target member 10.

Herein, the holder body 210 may include a mounting part 212 coupled to the first part 342-2 and a plurality of leg parts 214 extending from the mounting part 212, and the plurality of leg parts 214 may extend from an edge of the mounting part 212 so as to be spaced apart from each other along an edge of the mounting part 212. In addition, the plurality of gripping members 220 may be coupled to the plurality of leg parts 214 so as to correspond one-to-one.

Accordingly, when each of the plurality of gripping members 220 is coupled to the plurality of leg parts 214 while holding the training target member 10, the training target member 10 may be fixed to the holder 200 while maintaining a predetermined shape.

Accordingly, when the position of the first part 342-2 is changed through the driving part 300, the holder 200 coupled to the first part 342-2 may be changed in position together with the first part 342-2, and the training target member 10 fixed to the holder 200 may also be changed in position together with the holder 200.

Through this, the user may change the position of the training target member 10 fixed to the holder 200 within the training space (S1) such that the training target member 10 implements a specific part of the target organ, thereby increasing the surgical skill for the specific part of the organ requiring training.

In this case, each of the plurality of gripping members 220 may be detachably coupled to the plurality of leg parts 214.

For example, each of the plurality of gripping members 220 may be detachably coupled to the plurality of leg parts 214 by using a magnetic force.

To this end, the holder 200 may include a plurality of first magnetic members 230 provided on each of the plurality of leg parts 214 and a plurality of second magnetic members 240 provided on each of the plurality of gripping members 220, as illustrated in FIG. 6.

Herein, each of the first magnet member 230 and the second magnet member 240 may be a permanent magnet.

Accordingly, each of the plurality of gripping members 220 may be detachably coupled to the plurality of leg parts 214 through the corresponding first magnet member 230 and the second magnet member 240.

Through this, the user may use the corresponding first magnet member 230 and the second magnet member 240 to fix the training target member 10 to the holder 200 or easily remove the training target member 10 fixed to the holder 200 from the holder 200.

In this case, the holder 200 may be detachably coupled to one side of the driving part 300 by using a magnetic force similar to the plurality of gripping members 220.

To this end, the endoscopic surgery training apparatus 1000 according to an embodiment of the present disclosure may include a third magnet member 360 provided on one side of the driving part 300, and a fourth magnet member 250 provided on the holder 200 to correspond to the third magnet member 360.

For example, as shown in FIG. 7, the third magnet member 360 may be provided on the first part 342-2 of the holder support member 342, and the fourth magnet member 250 may be provided on the mounting part 212.

Herein, each of the third magnet member 360 and the fourth magnet member 250 may be a permanent magnet.

Accordingly, the mounting part 212 of the holder 200 may be detachably coupled to the first part 342-2 of the holder support member 342 through the corresponding third magnet member 360 and the fourth magnet member 250.

Through this, the user may use the corresponding third magnet member 360 and the fourth magnet member 250 to fix the holder 200 to the first part 342-2 of the holder support member 342 or easily separate the holder 200 fixed to the first part 342-2 from the first part 342-2.

In the present disclosure, the holder 200 may be provided to have an appropriate shape depending on the type of the training target member 10 or the organ to be implemented.

Accordingly, the user may perform training by using various training target members 10 by appropriately replacing the holder 200 according to the type or shape of the training target member 10.

In this case, as shown in FIG. 7, the holder 200 may include a protrusion 215 that is formed to protrude from one surface of the mounting part 212, and the holder support member 342 may include a settling groove 342a formed at a position corresponding to the protrusion 215 such that the protrusion 215 can be accommodated.

For example, the settling groove 342a may be formed to be recessed to a certain depth in one surface of the first part 342-2.

Accordingly, when the holder 200 is coupled to the first part 342-2 of the holder support member 342, the protrusion 215 may be inserted into the settling groove 342a.

As a result, when the holder 200 is coupled to the first part 342-2, the holder 200 may be prevented from moving on the first part 342-2 through the mutual coupling of the settling groove 342a and the protrusion 215.

Through this, even if the position of the first part 342-2 is variously changed through the driving part 300, the holder 200 may maintain the initial coupling position with respect to the first part 342-2.

Accordingly, when the position of the first part 342-2 is changed through the driving part 300 while the holder 200 to which the training target member 10 is fixed is coupled to the first part 342-2, the training target member 10 fixed to the holder 200 may be accurately changed to a desired position through a change in the position of the first part 342-2.

The driving part 300 may be disposed to be positioned so as to be positioned within the training space (S1), and the position of the holder support member 342 may be changed in the training space (S1).

Accordingly, the holder 200 coupled to the holder support member 342 may be changed in position through the driving of the driving part 300.

Through this, the driving part 300 may change the training target member 10 fixed to the holder 200 to a specific simulation position of the organ for training within the training space (S1).

That is, the driving part 300 may change the training target member 10 fixed to the holder 200 to a specific position by changing the position of the holder 200 coupled to the holder support member 342, and a specific part of the organ may be implemented by the training target member 10 through a change in position of the training target member 10.

Accordingly, the user may change the position of the training target member 10 fixed to the holder 200 through the driving part 300 while the holder 200 is coupled to the holder support member 342, thereby allowing the training target member 10 to implement a specific part of the intended organ.

In this case, the endoscopic surgery training apparatus 1000 according to an embodiment of the present disclosure may implement all of various parts of an organ regardless of the shape of the organ.

To this end, the driving part 300 may change the position of the holder 200 coupled to the holder support member 342 in various ways within the training space S1) through a four-degree-of-freedom movement including a first linear movement, a first rotation, a second rotation and a second linear movement.

That is, the endoscopic surgery training apparatus 1000 according to an embodiment of the present disclosure may enable the holder support member 342 to perform a first linear movement and a second linear movement through the driving part 300, while also enabling a first rotation and a second rotation around different axes.

As a result, the endoscopic surgery training apparatus 1000 according to an embodiment of the present disclosure may change the position of the holder 200 coupled to the holder support member 342 in various ways through a combination of the first linear movement, the second linear movement, the first rotation and the second rotation of the holder support member 342 through the driving part 300.

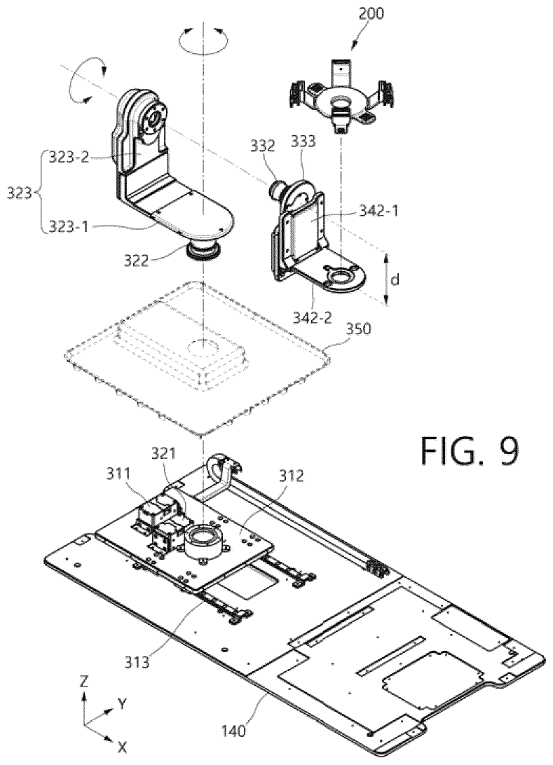

In this case, as shown in FIG. 9, the first part 342-2 of the holder support member 342 to which the holder 200 is coupled may be maintained in a state of being parallel to the second rotation axis 332, which is the central axis for the second rotation of the holder support member 342, and being spaced apart from the second rotation axis 332 by a certain distance (d).

Moreover, the first part 342-2 may move linearly along the X-axis direction together with the first rotation axis 322 and the second rotation axis 332 through the first linear movement, and the separation distance between the first part 342-2 and the second rotation axis 332, which is the central axis of the second rotation, may be adjusted through the second linear movement.

Accordingly, the rotation radius of the first part 342-2 centered on the second rotation axis 332 may be adjusted through the second linear movement of the first part 342-2, and when the first part 342-2 centered on the second rotation axis 332 is rotated at a certain angle, the rotation radius of the first part 342-2 centered on the first rotation axis 322 may be adjusted by the separation distance between the first part 342-2 and the second rotation axis 332.

Accordingly, when the position of the first part 342-2 of the holder support member 342 is changed by the driving part 300 while the holder 200 is coupled to the first part 342-2 of the holder support member 342, the training target member 10 fixed to the holder 200 may implement various parts of the organ regardless of the shape of the organ, and the user may implement a specific part of the desired organ similar to an actual environment by changing the position of the training target member 10 connected to the holder 200.

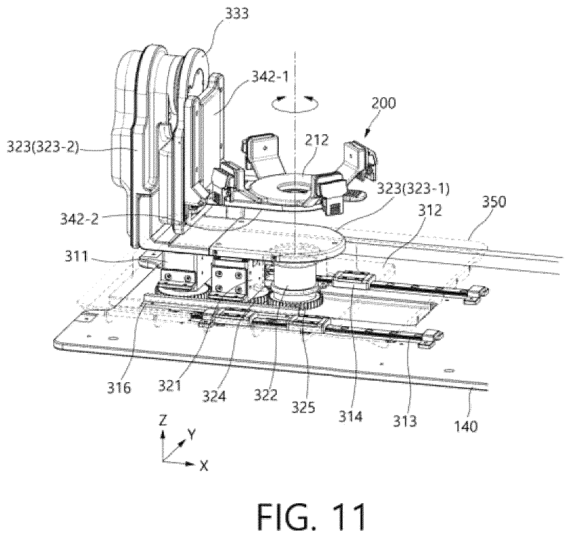

In addition, as shown in FIG. 11, when the holder support member 342 is placed at the initial position by the driving part 300 while the holder 200 is coupled to the holder support member 342, the center of the holder 200 may be arranged to be in a straight line with the first rotation axis 322, which is the central axis for the first rotation of the holder support member 342.

Herein, the center of the holder 200 may be a center point of the mounting part 212 or a center point of the protrusion 215.

Accordingly, when implementing various parts of an organ by changing the position of the training target member 10 fixed to the holder 200, the position of the holder 200 for implementing a specific part of the target organ may be easily calculated, and the first part 342-2 to which the training target member 10 is fixed may be rotated around the two rotation axes 322, 332 to implement the overall shape of the target organ.

Through this, the user may implement a position corresponding to a specific part of the overall shape of the target organ similar to an actual environment by changing the position of the training target member 10 coupled to the holder 200.

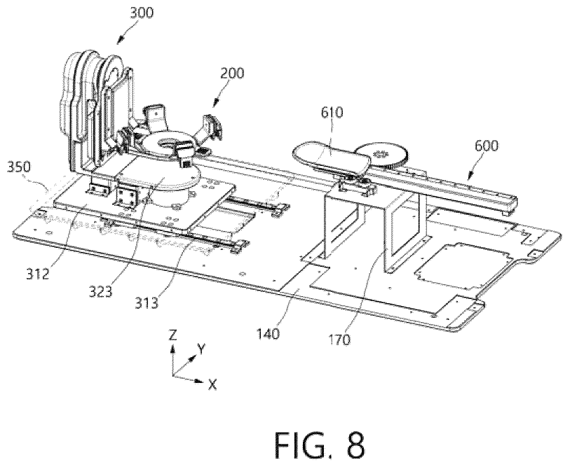

As a non-limiting example, the driving part 300 may include a first linear driving part 310 for the first linear movement of the holder support member 342, a first rotational driving part 320 for the first rotation of the holder support member 342, a second rotational driving part 330 for the second rotation of the holder support member 342, and a second linear driving part 340 for the second linear movement of the holder support member 342, as shown in FIGS. 8 to 14.

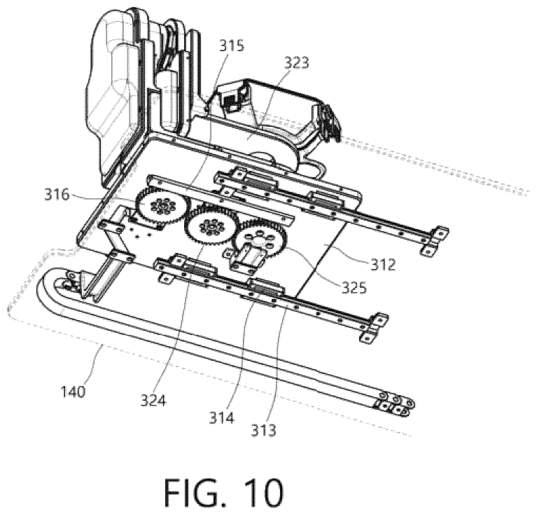

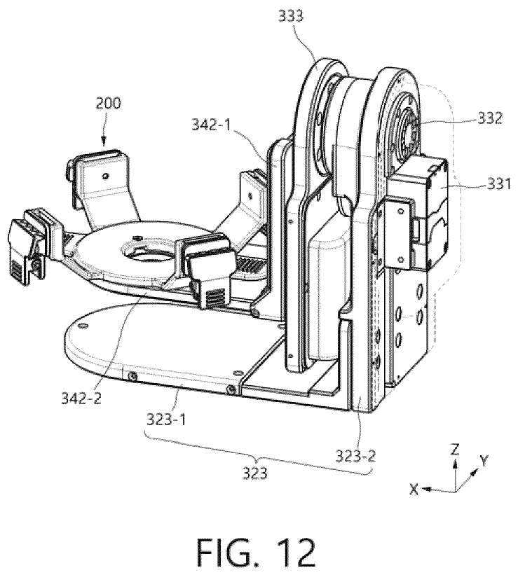

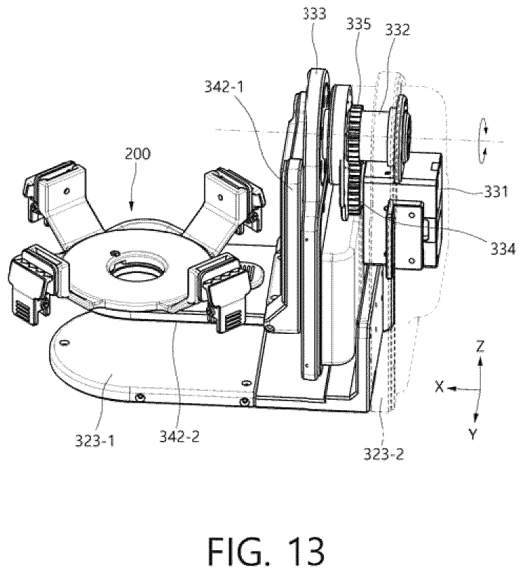

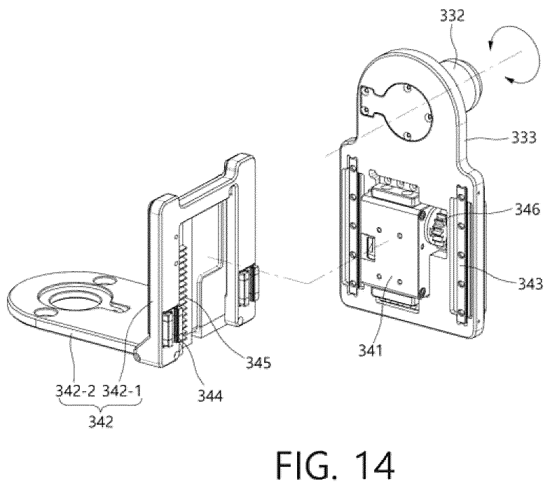

For reference, FIG. 8 is a drawing showing a support plate, a driving part and an auxiliary driving part extracted from the endoscopic surgery training apparatus according to an embodiment of the present disclosure, FIG. 9 is a drawing of a driving part and a holder extracted from the endoscopic surgical training apparatus according to an embodiment of the present disclosure, with some of the components separated, FIG. 10 is a drawing showing a lower part of a moving member in the endoscopic surgery training apparatus according to an embodiment of the present disclosure, FIG. 11 is a drawing showing the coupling relationship of a first rotational driving part in the endoscopic surgery training apparatus according to an embodiment of the present disclosure, FIG. 12 is a drawing showing the coupling relationship of a second rotational driving part in the endoscopic surgery training apparatus according to an embodiment of the present disclosure, FIG. 13 is a drawing showing FIG. 12 from another direction, showing a state in which the second mounting member is removed, and FIG. 14 is a drawing showing the coupling relationship of a second linear driving part in the endoscopic surgery training apparatus according to an embodiment of the present disclosure.

Specifically, the first linear driving part 310 may include a moving member 312 that reciprocates linearly along the X-axis direction with respect to a support plate 140 via a first driving motor 311, as illustrated in FIGS. 10 and 11.

Herein, the support plate 140 may be a plate-shaped member that forms a bottom surface of the training space (S1). In addition, the support plate 140 may be provided as a separate member from the first housing 110, or may be formed integrally with the first housing 110.

In this case, the first linear driving part 310 may further include a first guide rail 313 arranged along the X-axis direction, and a first slider 314 that is movably coupled to the first guide rail 313 so as to be linearly moved along the first guide rail 313.

In addition, the first linear driving part 310 may further include a first rack gear 315 and a first pinion gear 316 that is axially coupled with a rotation axis of the first driving motor 311 so as to be rotated by the first driving motor 311 and meshes with the first rack gear 315.

In this case, the first guide rail 313 may be fixed to one surface of the support plate 140 so as to be arranged parallel to the X-axis direction, the first slider 314 may be coupled to the first guide rail 313 so as to be linearly moved along the first guide rail 313, and the first driving motor 311 may be fixedly coupled to the moving member 312.

In addition, the first rack gear 315 may be fixedly coupled to one side of the moving member 312 so as to be arranged parallel to the X-axis direction, similar to the first guide rail 313.

Accordingly, when the first driving motor 311 rotates through the driving of the controller 500, the first pinion gear 316 may transmit a driving force to the first rack gear 315 through rotation, and the first rack gear 315 may convert a rotational force of the first pinion gear 316 into a driving force for linear movement.

That is, the first rack gear 315 may convert a rotational force of the first pinion gear 316 into a direction parallel to the X-axis.

Herein, the controller 500 may perform a role of controlling the overall driving and may be an MCU or a processor. Moreover, the controller 500 may further include a memory unit for storing input information.

Through this, the first slider 314 may reciprocally move linearly along the first guide rail 313 through the driving force provided from the first rack gear 315.

As a result, the moving member 312 to which the first slider 314 is fixed on one side may move linearly along the X-axis direction with respect to the support plate 140. That is, the first linear driving part 310 may implement the first linear movement of the moving member 312.

Referring again to FIGS. 10 and 11, the first rotational driving part 320 may include a second driving motor 321, a first rotational axis 322 that rotates by receiving a rotational force of the second driving motor 321, and a first mounting member 323 that is fixedly coupled to one end side of the first rotational axis 322.

In addition, the first rotational driving part 320 may further include a first driving gear 324 that is axially coupled to a rotational axis of the second driving motor 321), and a first driven gear 325 that is axially coupled to the first rotational axis 322 and meshes with the first driving gear 324.

In this case, the second driving motor 321 may be fixedly coupled to the moving member 312, and the first rotation axis 322 may be arranged parallel to the Z-axis, which is perpendicular to the X-axis, with respect to the moving member 312.

In addition, the first rotation axis 322 may be arranged parallel to the Z-axis such that the center of the holder 200 can be located in a straight line when the holder support member 342 is placed at the initial position while the holder 200 is coupled to the holder support member 342.

Moreover, the first driving gear 324 and the first driven gear 325 may be arranged to be positioned on a lower side of the moving member 312, and the first mounting member 323 fixed to one end side of the first rotational axis 322 may be arranged to be positioned on an upper side of the moving member 312.

Accordingly, when the second driving motor 321 rotates through the driving of the controller 500, the first driving gear 324 may rotate the first driven gear 325, and the first rotational axis 322 may rotate together with the first driven gear 325.

That is, the first rotation axis 322 may rotate around the Z axis that is perpendicular to the X axis with respect to the moving member 312, and the first mounting member 323 fixed to an end side of the first rotation axis 322 may rotate in the same direction as the first rotation axis 322.

As a result, the first mounting member 323 may rotate around the Z-axis that is perpendicular to the X-axis with respect to the moving member 312 through the rotation of the first rotation axis 322. That is, the first mounting member 323 may rotate around the first rotation axis 322 that is arranged parallel to the Z-axis, and the first rotational driving part 320 may implement a first rotation of the first mounting member 323 around the first rotation axis 322.

Herein, the endoscopic surgery training apparatus 1000 according to an embodiment of the present disclosure may further include a cover member 350 coupled to the moving member 312, and the cover member 350 may be coupled to the moving member 312 so as to cover the first driving motor 311, the second driving motor 321 and the first rotational axis 322 arranged on an upper surface of the moving member 312.

In this case, the first rotational axis 322 may be arranged to penetrate the cover member 350, and the first mounting member 323 may be coupled to the first rotational axis 322 so as to be exposed to the outside from an upper side of the cover member 350. Accordingly, the first mounting member 323 may freely rotate through a rotation of the first rotational axis 322.

Referring again to FIGS. 12 and 13, the second rotational driving part 330 may include the third driving motor 331, a second rotational axis 332 that rotates by receiving a rotational force of the third driving motor 331, and a second mounting member 333 that is fixedly coupled to the second rotational axis 332.

In addition, the second rotational driving part 330 may further include a second driving gear 334 that is axially coupled to a rotational axis of the third driving motor 331, and a second driven gear 335 that is axially coupled to the second rotational axis 332 and meshes with the second driving gear 334.

In this case, the third driving motor 331 may be fixedly coupled to the first mounting member 323, and the second rotation axis 332 may be arranged to be parallel to the XY plane that is perpendicular to the Z-axis, and to be perpendicular to the Z-axis.

In addition, one end part of the second rotation axis 332 may be rotatably coupled to the first mounting member 323, and the other end part of the second rotation axis 332 may be fixed to the second mounting member 333.

Herein, the first mounting member 323 may include a first-1 mounting member 323-1 to which an end of the first rotation axis 322 is fixed, and a first-2 mounting member 323-2 to which an end of the second rotation axis 332 is rotatably coupled.

In this case, the part where the end of the first rotation axis 322 is fixed in the first-1 mounting member 323-1 may be a part that is arranged parallel to the XY plane and perpendicular to the Z-axis, and the part where the end of the second rotation axis 332 is coupled in the first-2 mounting member 323-2 may be a part that is arranged parallel to the Z-axis.

In addition, the second mounting member 333 may be arranged parallel to the first-2 mounting member 323-2 with a gap therebetween, and the third driving motor 331 may be fixed to the first-2 mounting member 323-2.

Accordingly, the second rotation axis 332 may be arranged parallel to the XY plane and perpendicular to the Z-axis, with both ends coupled to the second mounting member 333 and the first-2 mounting member 323-2, respectively.

In addition, the first-1 mounting member 323-1 may be maintained in a state of being spaced apart from the second rotation axis 332 by a certain distance along the Z-axis direction while being parallel to the second rotation axis 332.

Through this, when the third driving motor 331 rotates through the driving of the controller 500, the second driving gear 334 may rotate the second driven gear 335, and the second rotation axis 332 may rotate together with the second driven gear 335.

That is, the second rotation axis 332 may rotate about an axis that is parallel to the XY plane and perpendicular to the Z-axis with respect to the first-2 mounting member 323-2, and the second mounting member 333 fixed to the second rotation axis 332 may rotate in the same direction as the second rotation axis 332.

As a result, the second mounting member 333 may rotate around an axis that is parallel to the XY plane and perpendicular to the Z axis through the rotation of the second rotation axis 332. That is, the second mounting member 333 may rotate around the second rotation axis 332 that is arranged parallel to the XY plane and perpendicular to the Z axis, and the second rotational driving part 330 may implement a second rotation of the second mounting member 333 around the second rotation axis 332.

In other words, the second mounting member 333 may rotate around the second rotation axis 332 that is arranged perpendicular to the first-2 mounting member 323-2.

Referring again to FIG. 14, the second linear driving part 340 may include a fourth driving motor 341 and a holder support member 342 that reciprocally moves linearly with respect to the second mounting member 333 through the fourth driving motor 341.

In addition, the second linear driving part 340 may further include a second guide rail 343 and a second slider 344 that is movably coupled to the second guide rail 343 so as to reciprocally move linearly along the second guide rail 343.

In this case, the second guide rail 343 may be arranged perpendicular to the second rotation axis 332 that is arranged parallel to the XY plane and perpendicular to the Z axis.

Moreover, the second linear driving part 340 may further include a second rack gear 345 arranged parallel to the longitudinal direction of the second guide rail 343, and a second pinion gear 346 that is axially coupled to a rotation axis of the fourth driving motor 341 so as to be rotated by the fourth driving motor 341 and meshes with the second rack gear 345.

In this case, the fourth driving motor 341 may be fixedly coupled to the second mounting member 333, and the second guide rail 343 may be fixed to one surface of the second mounting member 333 so as to be arranged perpendicular to the second rotation axis 332.

In addition, the second slider 344 may be fixed to one surface of the holder support member 342, the second slider 344 may be coupled to the second guide rail 323 so as to move linearly along the second guide rail 343, and the second rack gear 345 may be coupled to one side of the holder support member 342 so as to be arranged parallel to the longitudinal direction of the second guide rail 343 and mesh with the second pinion gear 346.

Herein, as described above, the holder support member 342 may include a second part 342-1 to which the second slider 344 and the second rack gear 345 are fixed, and a first part 342-2 to which the holder 200 is detachably coupled.

In addition, the second part 342-1 may be arranged such that one surface faces the second mounting member 333 as shown in FIG. 12 and FIG. 13, and the first part 342-2 may extend a certain length from the end of the second part 342-1 along a direction parallel to the axial direction of the second rotation axis 332.

That is, the second part 342-1 and the first part 342-2 may be arranged so as to be perpendicular to each other, and the first part 342-2 may be arranged so as to be parallel to the second rotation axis 332. In this case, the third magnet member 360 may be provided on the first part 342-2.

Accordingly, when the holder support member 342 is placed in the initial position through the driving part 300 as shown in FIG. 8, the first part 342-2 may be positioned above the moving member 312 and above the first-1 mounting member 323-1, and may be arranged parallel to both of the moving member 312 and the first-1 mounting member 323-1.

Likewise, when the holder support member 342 is placed in the initial position through the driving part 300 as shown in FIG. 8, the mounting part 212 of the holder 200 coupled to the first part 342-2 may also be positioned above the moving member 312 and above the first-1 mounting member 323-1, and may be arranged parallel to both of the moving member 312 and the first-1 mounting member 323-1.

Accordingly, the first part 342-2 may be arranged parallel to the second rotation axis 332 and spaced apart from the second rotation axis 332 by a certain distance (d) as shown in FIG. 9.

As a result, when the fourth driving motor 341 rotates by driving the controller 500, the second pinion gear 346 may transmit a driving force to the second rack gear 345 through rotation, and the second rack gear 345 may convert a rotational force of the second pinion gear 346 into a driving force for linear movement.

That is, the second rack gear 345 may convert a rotational force of the second pinion gear 346 into a direction parallel to the longitudinal direction of the second guide rail 343.

Through this, the second slider 344 may reciprocally move linearly along the second guide rail 343 through a driving force provided from the second rack gear 345. That is, the second slider 344 may reciprocally move linearly along the second guide rail 343 in a direction perpendicular to the second rotation axis 332.

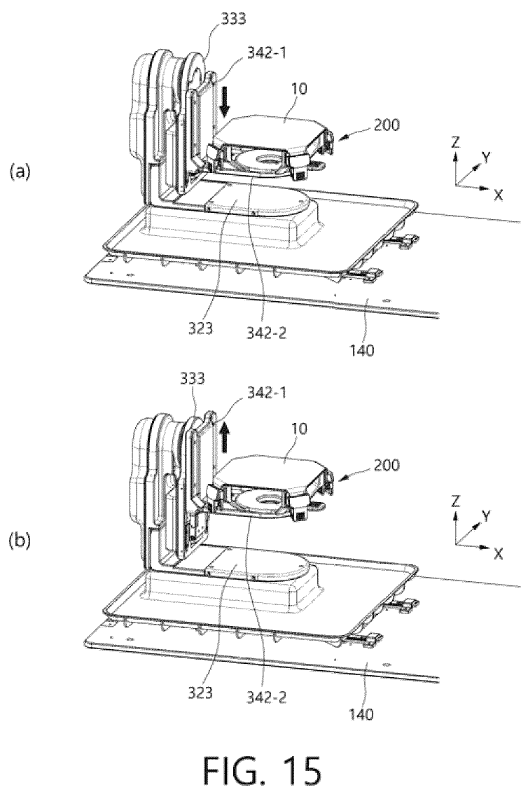

As a result, the holder support member 342 to which the second slider 344 is fixed on one side may reciprocally move linearly along the second guide rail 343 with respect to the second mounting member 333, as shown in FIG. 15. For reference, FIG. 15 is a drawing showing a state in which a holder support member moves linearly through a second linear driving part in the endoscopic surgery training apparatus according to an embodiment of the present disclosure.

In other words, the holder support member 342 may reciprocally move along a direction perpendicular to an axis that is parallel to the XY plane and perpendicular to the Z-axis. That is, the holder support member 342 may reciprocally move along a direction perpendicular to the second rotation axis 332 along the second guide rail 343 via the second slider 344.

Accordingly, the holder support member 342 may always move along a direction perpendicular to the second rotation axis 332 regardless of the rotation of the second mounting member 333.

That is, the second linear driving part 340 may implement a second linear movement of the holder support member 342.

Through this, the holder 200 coupled to the holder support member 342 and the training target member 10 mounted on the holder 200 may always move in a straight line along a direction perpendicular to the second rotation axis 332 regardless of the rotation of the second mounting member 333 together with the holder support member 342.

In other words, the first part 342-2 coupled to the holder 200 may always move in a straight line along a direction perpendicular to the second rotation axis 332 regardless of the rotation of the second mounting member 333 together with the second part 342-2.

Accordingly, the second linear driving part 340 may adjust a gap between the second rotation axis 332 and the first part 342-2 while maintaining the first part 342-2 and the second rotation axis 332 in a parallel state regardless of the rotation of the second mounting member 333 centered on the second rotation axis 332.

Through this, the second linear driving part 340 may adjust a rotation radius of the first part 342-2 centered on the second rotation axis 332 through the second linear movement of the first part 342-2.

In this way, in the endoscopic surgery training apparatus 1000 according to an embodiment of the present disclosure, the driving part 300 may be coupled such that the first linear driving part 310, the first rotational driving part 320, the second rotational driving part 330 and the second linear driving part 340 are interconnected, and the holder 200 may be mounted on the holder support member 342 constituting the second linear driving part 340.

Accordingly, the holder 200 may move in the same manner as the holder support member 342 through the driving of the driving part 300.

Through this, when the first linear driving part 310 is driven, if the moving member 312 moves linearly along the X-axis direction with respect to the support plate 140 together with the first slider 314, the holder 200 mounted on the holder supporting member 342 may move linearly along the X-axis direction with respect to the support plate 140 together with the holder supporting member 342. That is, the holder 200 mounted on the holder supporting member 342 may implement a first linear movement through the linear movement of the moving member 312 by the first linear driving part 310.

In addition, when the first rotational driving part 320 is driven, if the first mounting member 323 rotates about the first rotation axis 322 relative to the moving member 312, the holder 200 mounted on the holder support member 342 may rotate about the first rotation axis 322 together with the first mounting member 323. That is, the holder 200 mounted on the holder support member 342 may implement a first rotation through the rotation of the first mounting member 323 by the first rotational driving part 320.

Moreover, when the second rotational driving part 330 is driven, if the second mounting member 333 rotates about the second rotation axis 332 relative to the first mounting member 323, the holder 200 mounted on the holder support member 342 may rotate about the second rotation axis 332 together with the second mounting member 333. That is, the holder 200 mounted on the holder support member 342 may implement a second rotation through the rotation of the second mounting member 333 by the second rotational driving part 330.

In addition, when the second linear driving part 340 is driven, if the holder support member 342 moves linearly along a direction perpendicular to the second rotation axis 332 together with the second slider 344, the holder 200 coupled to the holder support member 342 may move linearly along a direction perpendicular to the second rotation axis 332 together with the holder support member 342. That is, the holder 200 mounted on the holder support member 342 may implement a second linear movement through the linear movement of the holder support member 342 by the second linear driving part 340.

Through this, the holder 200 mounted on the holder support member 342 in the endoscopic surgery training apparatus 1000 according to an embodiment of the present disclosure may implement a four-degree-of-freedom movement through the driving of the driving part 300.

That is, in the endoscopic surgery training apparatus 1000 according to an embodiment of the present disclosure, the holder 200 mounted on the holder support member 342 may perform a first linear movement through the first linear driving member 310, a first rotation through the first rotation driving member 320, a second rotation through the second rotation driving member 330, and a second linear movement through the second linear driving member 340.

In addition, when the holder support member 342 is placed at the initial position through the driving part 300 as described above, the center of the holder 200 may be placed so as to be in a straight line with the first rotation axis 322, which is the central axis for the first rotation of the holder support member 342.

Moreover, the first part 342-2 of the holder support member 342 to which the holder 200 is coupled may be maintained in a state of being spaced apart in parallel with the second rotation axis 332, which is the central axis for the second rotation of the holder support member 342, while the distance from the second rotation axis 332 may be adjusted.

Accordingly, the endoscopic surgery training apparatus 1000 according to an embodiment of the present disclosure may implement all four degrees of freedom of movement of the holder 200 mounted on the holder support member 342, thereby allowing the position of the holder 200 mounted on the holder support member 342 to be changed in various ways through a combination of the first linear movement of the holder support member 342 by the first linear driving part 310, the second linear movement of the holder support member 342 by the second linear driving part 340, the first rotation of the holder support member 342 by the first rotational driving part 320, and the second rotation of the holder support member 342 by the second rotational driving part 330.

Accordingly, when the position of the first part 342-2 is changed by the driving part 300 while the holder 200 is coupled to the first part 342-2 of the holder support member 342, the training target member 10 fixed to the holder 200 may implement all of various parts of the organ regardless of the shape of the organ, and the user may implement a specific part of the target organ similar to the actual environment by changing the position of the training target member 10 coupled to the holder 200.

That is, in the endoscopic surgery training apparatus 1000 according to an embodiment of the present disclosure, the training target member 10 fixed to the holder 200 may implement all of various parts of the organ regardless of the shape of the organ, and the user may implement a specific part of the target organ similar to the actual environment through the training target member 10.

For example, as shown in FIG. 8 and FIG. 11, the holder 200 may be mounted on the holder support member 342 of the driving part 300, and the holder support member 342 may be disposed to be positioned at the initial position through the driving part 300.

Herein, when the holder support member 342 is disposed to be positioned at the initial position, the first slider 314 may be moved to the leftmost position along the first guide rail 313, the rotation angle of the first mounting member 323 and the rotation angle of the second mounting member 333 may be 0 degrees, and the second slider 344 may be positioned as far away from the second rotation axis 332 as possible along the second guide rail 343.

In addition, the training target member 10 for implementing the stomach may be mounted on the holder 200.

Then, while the training target member 10 is mounted on the holder 200, the first mounting member 323 may be rotated 105 degrees clockwise around the first rotation axis 322 through the first rotation drive member 320, and the second mounting member 333 may be rotated 21 degrees clockwise around the second rotation axis 332 through the second rotation drive member 330.

Thereafter, the holder support member 342 may be moved linearly 36 mm in a direction closer to the second rotation axis 332 through the second linear drive member 340.

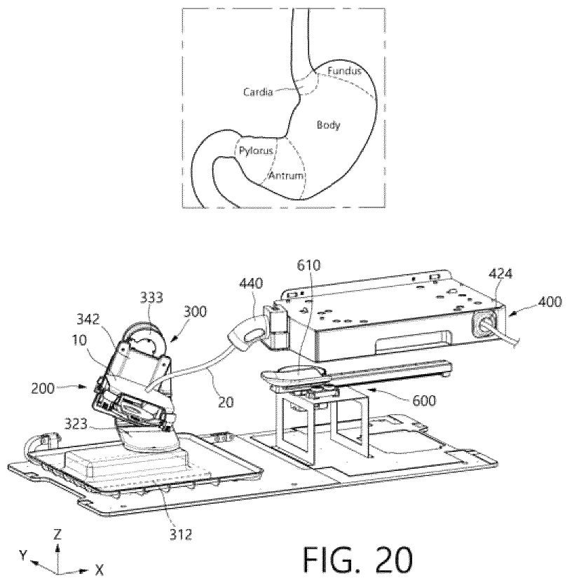

Through this, the holder 200 mounted on the holder support member 342 may be rotated 105 degrees clockwise around the first rotation axis 322 as shown in FIG. 20, rotated 21 degrees clockwise around the second rotation axis 332, and then moved 36 mm in a straight line in a direction closer to the second rotation axis 332.

For reference, FIG. 20 is a drawing showing a state in which the endoscopic surgery training apparatus according to an embodiment of the present disclosure is in use, showing a state in which a holder and a training target member are disposed to be positioned at different specific positions.

As a result, the holder 200 mounted on the holder support member 342 may be changed to a first simulation position corresponding to the greater curvature (GC) part of the antrum of the stomach in the training space (S1), and the training target member 10 mounted on the holder 200 changed to the first simulation position in the training space (S1) may implement the inner wall of the GC part of the antrum of the stomach.

That is, the endoscopic surgery training apparatus 1000 according to an embodiment of the present disclosure may implement the greater curvature (GC) part of the antrum of the stomach in the training space (S1) by changing the holder 200 mounted on the holder support member 342 to the first simulation position, and the training target member 10 mounted on the holder 200 may implement the inner wall of the GC part of the antrum of the stomach in the first simulation position.

As another example, as shown in FIG. 8 and FIG. 11, the holder 200 may be mounted on the holder support member 342 of the driving part 300, and the holder support member 342 may be disposed so as to be positioned at the initial position described above, and the training target member 10 for implementing camouflage may be mounted on the holder 200.

Then, the first mounting member 323 may be rotated 75 degrees counterclockwise around the first rotation axis 322 through the first rotation driving member 320, and the second mounting member 333 may be rotated 133 degrees clockwise around the second rotation axis 332 through the second rotation driving member 330.

Afterwards, the holder support member 342 may be linearly moved 28.5 mm in a direction closer to the second rotation axis 332 through the second linear driving member 340.

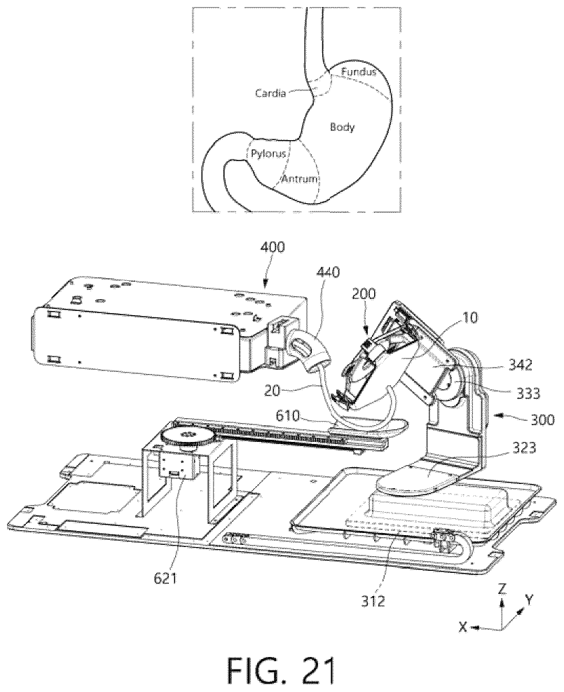

Through this, while the holder 200 mounted on the holder support member 342 is rotated 75 degrees clockwise around the first rotation axis 322 as shown in FIG. 21, it may be rotated 133 degrees counterclockwise around the second rotation axis 332 and then linearly moved 28.5 mm in a direction closer to the second rotation axis 332.

As a result, the holder 200 mounted on the holder support member 342 may be changed to a second simulation position corresponding to the lesser curvature (LC) part of the antrum of the stomach in the training space (S1), and the training target member 10 mounted on the holder 200 changed to the second simulation position in the training space (S1) may implement the inner wall of the LC part of the antrum of the stomach.

That is, the endoscopic surgery training apparatus 1000 according to an embodiment of the present disclosure may implement the lesser curvature (LC) part of the antrum of the stomach in the training space (S1) by changing the holder 200 mounted on the holder support member 342 to the second simulation position, and the training target member 10 mounted on the holder 200 may implement the inner wall of the LC part of the antrum of the stomach in the second simulation position.

As another example, as shown in FIG. 8 and FIG. 11, the holder 200 may be mounted on the holder support member 342 of the driving part 300, the holder support member 342 may be disposed to be positioned at the initial position described above, and the training target member 10 for implementing the colon may be mounted on the holder 200.

Then, the moving member 312 may be moved 61 mm along the X-axis direction through the first linear driving part 310, and the first mounting member 323 may be rotated 15 degrees clockwise around the first rotation axis 322 through the first rotational driving part 320.

Thereafter, the second mounting member 333 may be rotated 10 degrees clockwise around the second rotation axis 332 through the second rotation driving member 330, and the holder support member 342 may be linearly moved 15 mm in a direction closer to the second rotation axis 332 through the second linear driving member 340.

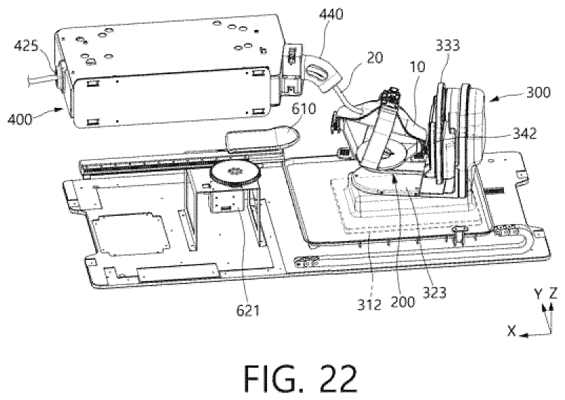

Through this, the holder 200 mounted on the holder support member 342 may be linearly moved 61 mm along the X-axis direction as shown in FIG. 22, rotated 15 degrees clockwise around the first rotation axis 322, rotated 10 degrees clockwise around the second rotation axis 332, and then linearly moved 15 mm in a direction closer to the second rotation axis 332.

As a result, the holder 200 mounted on the holder support member 342 may be changed to a third simulation position corresponding to the Downside part of the colon in the training space (S1), and the training target member 10 mounted on the holder 200 may implement the inner wall of the Downside part of the colon.

That is, the endoscopic surgery training apparatus 1000 according to an embodiment of the present disclosure may implement the Downside part of the colon in the training space (S1) by changing the holder 200 mounted on the holder support member 342 to the third simulation position, and the training target member 10 mounted on the holder 200 may implement the inner wall of the Downside part of the colon in the third simulation position.

In this way, the endoscopic surgery training apparatus 1000 according to an embodiment of the present disclosure may implement a specific part of a target organ in the training space (S1) similar to the real one by changing the simulation position of the holder 200 through the training target member 10 fixed to the holder 200 and the driving part 300.

Therefore, the user may conduct training to increase skill by using the training target member 10 implemented in the training space (S1) to simulate a specific part of a target organ.

In this case, the controller 500 may drive the driving part 300 such that the movement of the holder support member 342 can occur in a state where the training target member 10 mounted on the holder 200 is changed to a specific simulation position of the organ for training in the training space (S1) (e.g., one of the first simulation position, the second simulation position and the third simulation position).

In other words, the controller 500 may control the driving part 300 to perform a simulation operation that simulates the movement of an organ that can occur during an endoscopic surgery process while the training target member 10 is changed to a specific simulation position of an organ for training in the training space (S1).

The movements of an organ that can occur during an actual endoscopic surgery process are as follows.

For example, when air is injected into the interior of an organ during an actual endoscopic surgery process, the organ may expand and move such that the tip of the endoscopic device and the inner wall of the organ move away from each other.

For example, when air is sucked into the interior of an organ during an actual endoscopic surgery process, the organ may contract and move such that the tip of the endoscopic device and the inner wall of the organ move closer.

For example, an organ may move due to the patient's breathing during an actual endoscopic surgery process.

For example, an organ may move due to an event such as a patient's gagging or sneezing during an actual endoscopic surgery process.

The controller 500 may control at least one of the first linear driving part 310, the first rotational driving part 320, the second linear driving part 340 and the second rotational driving part 330 such that the training target member 10 performs a simulation movement that simulates the movement of an organ that may occur during the endoscopic surgery process described above.

For example, the controller 500 may drive the second linear driving part 340 such that the holder support member 342 moves linearly along a direction perpendicular to the second rotation axis 332 in a state where the training target member 10 mounted on the holder 200 is changed to a specific simulation position of an organ for training in the training space (S1) (e.g., one of the first simulation position, the second simulation position and the third simulation position).

Accordingly, the training target member 10 mounted on the holder 200 may perform a simulation movement of moving in a straight line along a direction perpendicular to the second rotation axis 332 together with the holder support member 342 by moving the holder support member 342.

Through this, when the tip of the endoscope device 20 is arranged on one side of the training target member 10 as shown in FIGS. 20 to 22, the training target member 10 may move in a direction closer to or further away from the tip of the endoscope device 20.

For reference, FIG. 21 is a drawing showing a state in which the endoscopic surgery training apparatus according to an embodiment of the present disclosure is in use, showing a state in which a holder and a training target member are disposed to be positioned at different specific positions, and FIG. 22 is a diagram showing a state in which the endoscopic surgery training apparatus according to an embodiment of the present disclosure is in use, showing a state in which a holder and a training target member are disposed to be positioned at other specific positions.

The movement of the training target member 10 moving in a straight line in a direction closer to the tip of the endoscope device 20 simulates the movement of injecting air into the interior of an organ during an actual endoscopic surgery to expand the organ, thereby bringing the tip of the endoscope device and the inner wall of the organ closer.

In addition, the movement of the training target member 10 moving in a straight line away from the tip of the endoscope device 20 simulates the movement of the tip of the endoscope device and the inner wall of the organ moving away by inhaling air inside the organ and contracting the organ during actual endoscopic surgery.

As a result, the endoscopic surgery training apparatus 1000 according to an embodiment of the present disclosure may identically implement an air-inflation/deflation situation in which air is injected into the inside of an organ during actual endoscopic surgery to inflate or contract the organ by allowing the training target member 10 mounted on the holder 200 to move in a straight line through the second linear driving part 340 while the training target member 10 is changed to a specific simulated position of the organ for training in the training space (S1).

In this case, the controller 500 may control the driving part 300 such that at least one of the first linear movement by the first linear driving part 310, the first rotation by the first rotational driving part 320, and the second rotation by the second rotational driving part 330 can be performed together with the second linear movement by the second linear driving part 340 in a state where the training target member 10 mounted on the holder 200 is changed to a specific simulation position of an organ for training in the training space (S1).

Accordingly, the training target member 10 mounted on the holder 200 may be moved in a specific simulation position of an organ for training in the training space (S1) by the driving part 300 not only in a straight line but also in a normal direction or in a twisted direction, thereby implementing a more diverse air-inflation/deflation situation.

As another example, in the endoscopic surgery training apparatus 1000 according to an embodiment of the present disclosure, the controller 500 may control the driving part 300 such that the holder support member 342 may perform at least one movement among a first linear movement by the first linear driving part 310, a first rotation by the first rotational driving part 320, a second rotation by the second rotational driving part 330, and a second linear movement by the second linear driving part 340 while the training target member 10 mounted on the holder 200 is changed to a specific simulated position of an organ for training in the training space (S1).

Accordingly, the training target member 10 mounted on the holder 200 may perform a simulated movement together with the holder support member 342 through the movement of the holder support member 342.

In this case, with the tip of the endoscope device 20 placed on one side of the training target member 10, the training target member 10 may perform a movement that simulates the movement of an organ due to a patient's breathing or an event (ex. coughing, sneezing) during an actual endoscopic surgery process through the controller 500.

Therefore, the endoscopic surgery training apparatus 1000 according to an embodiment of the present disclosure may similarly implement the movement of an organ that occurs in various situations, such as breathing, nausea, sneezing and the like that may occur during an endoscopic surgery process, through the controller 500 in a state where the training target member 10 mounted on the holder 200 is changed to a specific simulated position of an organ for training in the training space (S1).

In the present disclosure, the movement of the training target member 10 mounted on the holder 200 through the driving part 300 in a state where the training target member 10 is changed to a specific simulation position of an organ for training in the training space (S1) may be performed by the controller 500 controlling the driving part 300 when the user operates the same, or may be performed by the controller 500 automatically controlling the driving part 300 based on input data.

The channel forming part 400 may form an entry path for an endoscopic device 20 entering the training space (S1) from the outside.

That is, as shown in FIGS. 20 to 22, the tip of the endoscope device 20 may enter the training space (S1) after passing through the channel forming part 400, and may move toward the training target member 10 fixed to the holder 200 in the training space (S1).

Accordingly, the endoscope device 20 that has entered the training space (S1) through the channel forming part 400 may be disposed such that the tip faces one surface of the training target member 10 that has been changed to a specific simulation position of an organ for training in the training space (S1).

Through this, the user may train endoscopic surgery in the training space (S1) by bringing the tip of the endoscope device 20 close to the training target member 10 that has been changed to a specific simulation position of an organ for training in the training space (S1).

In this case, the channel forming part 400 may form at least two entry paths.

For example, the channel forming part 400 may form a first entry path that simulates the entry path of the endoscope device 20 during an upper gastrointestinal endoscopy and a second entry path that simulates the entry path of the endoscope device 20 during a colonoscopy.

To this end, as shown in FIGS. 1 to 4, FIGS. 16 and 17, the channel forming part 400 may include a channel case 410 coupled to the housing 100, a path part 420 formed by being formed to be recessed in from one surface of the channel case 410 so as to form an entry path of the endoscope device 20, and a path maintenance member 430 formed in a hollow shape such that the tip of the endoscope device 20 can pass through.

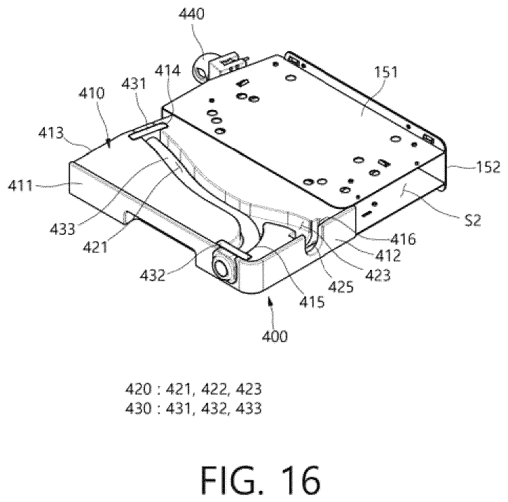

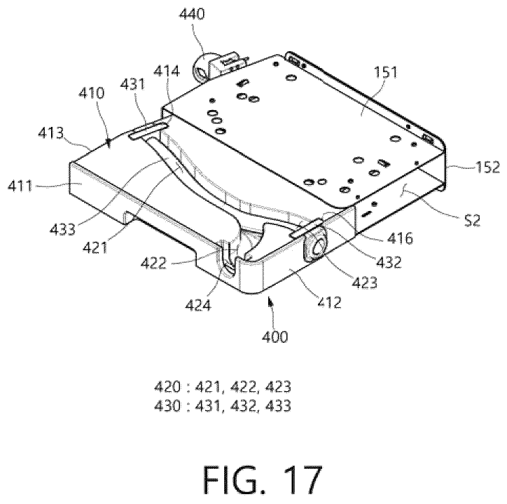

For reference, FIG. 16 is a drawing showing a channel forming part that is applicable to the endoscopic surgery training apparatus according to an embodiment of the present disclosure, showing a state in which a path maintenance member is mounted to be positioned at the first path forming part and the second path forming part, and FIG. 17 is a drawing showing a channel forming part that is applicable to the endoscopic surgery training apparatus according to an embodiment of the present disclosure, showing a state in which a path maintenance member is mounted to be positioned at the first path forming part and the third path forming part.

In this case, the path part 420 may include a first path forming part 421 formed such that one end is in communication with the training space (S1) and has a certain length, and a second path forming part 422 and a third path forming part 423 respectively extending from the first path forming part 421 so as to branch in different directions from the other end of the first path forming part 421.

In addition, the channel forming part 400 may include an exit forming member 440 coupled to the channel case 410 so as to be coupled to an end of the first path forming part 421, an end of the second path forming part 422 may be formed so as to be exposed to the outside to form a first entrance 424 through which the endoscope device 20 enters, and an end of the third path forming part 423 may also be formed so as to be exposed to the outside to form a second entrance 425 through which the endoscope device 20 enters.

Herein, the channel case 410 may include a first side surface 411 on which the first entrance 424 is formed, a second side surface 412 on which the second entrance 425 is formed, and a third side surface 413 on which the exit forming member 440 is coupled.

In addition, the first path forming part 421 and the second path forming part 422 may be arranged at a predetermined angle so as to simulate a path that is similar to a path connecting the throat and the esophagus through which the tip of the endoscope device 20 enters during an upper endoscopy, and the first path forming part 421 and the third path forming part 423 may be arranged to form an approximately straight line so as to simulate a path that is similar to a path connecting the anus and the rectum through which the tip of the endoscope device 20 enters during a colonoscopy.

Accordingly, when the path maintenance member 430 is disposed in the path part 420 so as to be positioned at the first path forming part 421 and the second path forming part 422 as shown in FIG. 16, the channel forming member 400 may form a first entry path that simulates the path through which the endoscope device 20 enters during an upper gastrointestinal endoscopy.

Through this, when the tip of the endoscope device 20 is inserted into the first entrance 424, the tip of the endoscope device 20 may move along the path maintenance member 430 forming the first entry path and then enter the training space (S1) through the exit forming member 440.

As a result, the operator may perform training in an environment similar to an actual gastroscopy by allowing the endoscope device 20 to enter the training space (S1) along a path similar to the path through which the endoscope device 20 enters during an actual gastroscopy through the channel forming part 400.

On the other hand, if the path maintenance member 430 is disposed in the path part 420 so as to be positioned at the first path forming part 421 and the third path forming part 423 as shown in FIG. 17, the channel forming part 400 may form a second entry path that simulates the path through which the endoscope device 20 enters during a colonoscopy.

Through this, when the tip of the endoscope device 20 is inserted into the second entrance 425, the tip of the endoscope device 20 may move along the path maintenance member 430 forming the second entry path and then enter the training space (S1) through the exit forming member 440.

As a result, the operator may perform training in an environment similar to an actual colonoscopy by allowing the endoscope device 20 to enter the training space (S1) along a path similar to the path through which the endoscope device 20 enters during an actual colonoscopy through the channel forming member 400.

In this case, the path maintenance member 430 may be detachably coupled to the path part 420, and the channel forming member 400 may be provided in a drawer-type manner in which the channel case 410 is slidably coupled to the housing 100 along the rail 180.

Through this, the user may change the position of the path maintenance member 430 coupled to the path part 420 while the path part 420 is exposed to the outside by withdrawing the channel case 410 from the housing 100.

Therefore, the user may easily change the entry path of the endoscope device 20 entering the training space (S1) from the outside.

That is, the user may pull out the channel case 410 from the housing 100 and mount the path maintenance member 430 on the path part 420 such that the path part 420 is exposed to the outside, or mount the path maintenance member 430 on the path part 420 such that the path maintenance member 430 is positioned on the first path forming part 421 and the third path forming part 423.

Through this, the entry path of the endoscope device 20 formed through the path maintenance member 430 may be easily changed.

Then, when the user changes the position of the path maintenance member 430 coupled to the path part 420 and inserts the channel case 410 into the interior of the housing 100, the path maintenance member 430 may selectively connect the first entrance 424 to the exit forming member 440 or the second entrance 425 to the exit forming member 440.

To this end, the channel forming member 400 may be slidably coupled to a storage space (S2) of the housing 100 by the channel case 410, and the path maintenance member 430 may be detachably coupled to the path part 420 through the first coupling port 431 and the second coupling port 432 provided on each end side, respectively.

For example, the housing 100 may include a storage space (S2) formed to be adjacent to the training space (S1), and the storage space (S2) may be defined by a space forming member 151 having a cross-sectional shape roughly in the shape of the letter ‘U’ with three open sides as shown in FIGS. 2, 15, and 16, and a finishing plate 152 covering one side of the space forming member 151. In addition, the exit forming member 440 may be coupled to the space forming member 151 so as to be in communication with the storage space (S2).

Through this, the storage space (S2) may be formed in the housing 100 such that two side surfaces are open.

That is, the storage space (S2) may be formed such that a first corresponding side surface corresponding to the first side surface 411 and a second corresponding side surface corresponding to the second side surface 412 are open when the channel case 410 is inserted into the storage space (S2).

Accordingly, even if the channel case 410 is inserted into the storage space (S2), the first entrance 424 and the second entrance 425 may remain exposed to the outside through the first corresponding side surface and the second corresponding side surface of the storage space (S2), respectively.

As a result, the endoscope device 20 may enter the training space (S1) through the first entrance 424 or the second entrance 425, and the channel case 410 may be inserted into the storage space (S2) or withdrawn from the storage space (S2) through the first corresponding side surface.

In this case, the channel case 410 may be guided to slide through the rail 180. For example, the rail 180 may be formed to correspond to one surface of the channel case 410 and one surface of the space forming member 151 facing each other.

In addition, the path maintenance member 430 may include a hollow connecting pipe 433 having a predetermined length, a first coupling port 431 provided at one end of the connecting pipe 433 so as to be detachably coupled to one end of the first path forming part 421, and a second coupling port 432 provided at the other end of the connecting pipe 433 so as to be detachably coupled to one end of the second path forming part 422 or one end of the third path forming part 423, and the connecting pipe 433 may be made of a material whose shape can be easily changed by an external force.

In this case, the channel case 410 may include a first coupling groove 414 formed to have a shape corresponding to the first coupling hole 431 while being positioned at the end side of the first path forming part 421, a second coupling groove 415 formed to have a shape corresponding to the second coupling hole 432 while being positioned at the end side of the second path forming part 422, and a third coupling groove 41) formed to have a shape corresponding to the second coupling hole 432 while being positioned at the end side of the third path forming part 423.

Accordingly, the first coupling port 431 may be detachably coupled to the first coupling groove 414, and when the first coupling port 431 is coupled to the first coupling groove 414, the connecting pipe 433 may be communicated with the exit forming member 440.

In addition, the second coupling port 432 may be detachably coupled to the second coupling groove 415 or the third coupling groove 416, and when the second coupling port 432 is coupled to the second coupling groove 415, the connecting pipe 433 may be communicated at one end with the first entrance 424, and when the second coupling port 432 is coupled to the third coupling groove 416, the connecting pipe 433 may be communicated at one end with the second entrance 425.

Accordingly, while the first coupling port 431 is coupled to the first coupling groove 414, when the second coupling port 432 is selectively coupled to either the second coupling groove 415 or the third coupling groove 416, the path maintenance member 430 may form a first entry path or a second entry path.

Meanwhile, the endoscopic surgery training apparatus 1000 according to an embodiment of the present disclosure may further include an auxiliary support part 600 for supporting a part of the endoscopic device 20 that has entered the training space (S1) through the channel forming member 400.

Such an auxiliary support part 600 may include an auxiliary support 610 for supporting a part of the endoscope device 20, and may be disposed so as to be positioned at a bottom of the channel forming member 400, and when necessary, the auxiliary support 610 may move linearly toward the holder 200 along the X-axis direction.

That is, the auxiliary support 610 may move linearly toward the holder 200 while being positioned at a bottom of the channel forming member 400 only when it is necessary to support a part of the endoscope device 20 that has entered the training space (S1) from the channel forming member 400.

For example, during an actual endoscopic surgery, the endoscope device 20 that has entered the inside of an organ may maintain a state in which a part (ex., a curved part excluding the tip) is in contact with the inner wall of the organ depending on a specific part of the organ.

That is, when the tip of the endoscope device 20 approaches a specific part of an organ, a portion of the entire length may be maintained in a bent state, and the bent portion may be maintained in a state of contacting the inner wall of the organ.

In the present disclosure, by supporting a portion of the endoscope device 20 that has entered the training space (S1) through the auxiliary support 610, a state in which a portion of the endoscope device 20 that has entered the interior of an organ during an actual endoscopic surgery contacts the inner wall of the organ may be similarly implemented.

For example, as shown in FIG. 21, when the training target member 10 mounted on the holder 200 implements the inner wall of the lesser curvature (LC) portion of the Antrum of the stomach in the training space (S1), the auxiliary support 610 may move 190 mm linearly toward the holder 200 along the X-axis direction while being positioned at a bottom of the channel forming part 400 by driving the controller 500.

Accordingly, the auxiliary support 610 may support the bent portion of the endoscope device 20 that entered the training space (S1) through the channel forming part 400, thereby implementing a state in which the bent portion of the endoscope device 20 that entered the inside of the stomach during an actual gastrointestinal endoscopy comes into contact with the inner wall of the greater curvature (GC) portion of the antrum of the stomach.

On the other hand, as shown in FIG. 20, when the training target member 10 mounted on the holder 200 implements the inner wall of the GC portion of the antrum of the stomach in the training space (S1), the auxiliary support 610 may maintain the initial state of being positioned at a bottom of the channel forming part 400 without entering the training space (S1) side through the driving of the controller 500.

Through this, the auxiliary support 610 may move to the training space (S1) through the controller 500 only when necessary according to a specific portion of the organ implemented through the training target member 10 in the training space (S1).

To this end, the auxiliary support part 600 may include an auxiliary support 610 for supporting a part of the endoscope device 20 that has entered the training space (S1), and an auxiliary driving part 620 for changing the position of the auxiliary support 610 through a fifth driving motor 621 within the training space (S1).

In this case, the controller 500 may drive the auxiliary driving part 620 to move the auxiliary support 610 toward the training target member 10 when the training target member 10 is changed to a specific simulation position of an organ that has been input in the training space (S1).

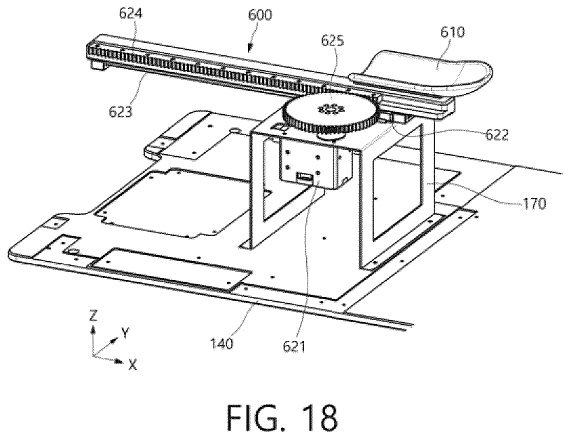

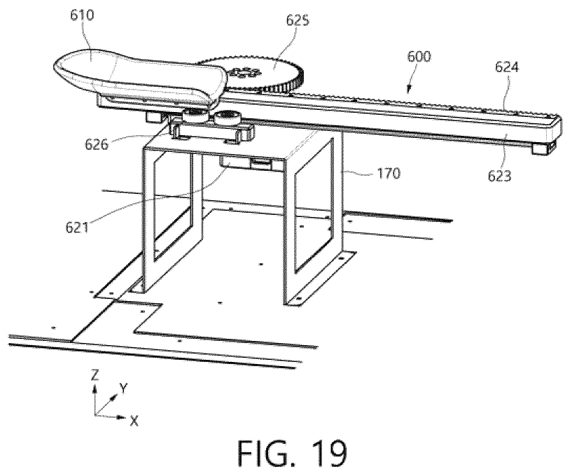

As a specific example, as shown in FIGS. 18 and 19, the auxiliary driving part 620 may include a third slider 622 fixed to the coupling member 170, and a third guide rail 623 movably coupled to the third slider 622 so as to be able to move linearly in the X-axis direction and having the auxiliary support 610 fixedly coupled to one side.

For reference, FIG. 18 is a drawing showing an auxiliary driving part that is applicable to the endoscopic surgery training apparatus according to an embodiment of the present disclosure, and FIG. 19 is a drawing looking at FIG. 18 from another direction.

The coupling member 170 may be coupled to the support plate 140, and the auxiliary driving part 620 may be spaced apart from the support plate 140 by a certain height such that the auxiliary driving part 620 may operate smoothly.

In addition, the auxiliary driving part 620 may further include a third rack gear 624 formed along the X-axis direction on one side of the third guide rail 623, and a third pinion gear 625 that is axially coupled to a rotation axis of the fifth driving motor 621 so as to be rotated by the fifth driving motor 621 and meshes with the third rack gear 624, and the fifth driving motor 621 may be fixedly coupled to the coupling member 170.

Moreover, the auxiliary driving part 620 may further include a guide roller 626 for guiding the sliding movement of the third guide rail 623, and the guide roller 626 may be coupled to the coupling member 170 so as to be positioned on the opposite side of the third rack gear 624 and contact one side of the third guide rail 623.

Accordingly, when the fifth driving motor 621 rotates through the driving of the controller 500, the third pinion gear 625 may transmit a driving force to the third rack gear 624 through rotation, and the third rack gear 624 may convert the rotational force of the third pinion gear 625 into a driving force for linear movement.

That is, the third rack gear 624 may convert the rotational power of the third pinion gear 625 in a direction parallel to the X-axis.

Through this, the third guide rail 623 may reciprocally move in a linear direction along the X-axis through the third slider 622 through the driving force provided by the third rack gear 624, and the third guide rail 623 may smoothly move in a linear direction through the guide roller 626.

As a result, the auxiliary support 610 fixed to one side of the third guide rail 623 may move in a linear direction along the X-axis to enter the training space (S1) and move toward the training target member 10 or maintain the initial state of being positioned at a bottom of the channel forming part 400.

Herein, the housing 100 may further include a placement space (S3) in which the auxiliary support part 600 is placed inside, and the placement space (S3) may be separated from the training space (S1) by a partition wall 160.

In addition, the placement space (S3) may be formed inside the housing 100 so as to be adjacent to the training space (S1) along the X-axis direction and positioned below the channel forming member 400.

Through this, when the training target member 10 fixed to the holder 200 is changed to a specific simulation position (e.g., the second simulation position) that requires the auxiliary support 610 among the specific simulation positions of an input organ, the controller 500 may drive the auxiliary driving part 620 to linearly move the auxiliary support 610 from the placement space (S3) to the training target member 10 arranged in the training space (S1).

On the other hand, when the training target member 10 fixed to the holder 200 is changed to a specific simulation position (e.g., the first simulation position or the third simulation position) that does not require the auxiliary support 610 among the specific simulation positions of an input organ, the controller 500 may maintain the auxiliary support 610 in the initial state of being placed in the placement space (S3).

Meanwhile, the controller 500 may control at least one of the first linear driving part 310, the first rotational driving part 320, the second linear driving part 340, the second rotational driving part 330 and the auxiliary driving part 620 such that the training target member 10 performs a simulation movement that simulates the movement of an organ that can occur in various situations such as breathing, gagging and sneezing during an endoscopic surgery process.

Meanwhile, the endoscopic surgery training apparatus 1000 according to an embodiment of the present disclosure may further include an operating part 700 for user operation.

Such an operating part 700 may generate a user input signal for changing the training target member 10 to a specific simulation position of an organ.

For example, the operating part 700 may be a known touch screen panel. In this case, the operating part 700 may be provided on one side of the housing 100 as shown in FIG. 1. Alternatively, the operating part 700 may be separated from the housing 100 and communicate with the controller 500 in a wired or wireless manner.

As another example, the operating part 700 may be a known terminal that is capable of wired or wireless communication, such as a smart phone or tablet PC.

A user may transmit an input signal to the controller 500 through the operating part 700, and the controller 500 may control the overall operation including the driving part 300 and the auxiliary driving part 620 based on the input signal transmitted from the operating part 700.

As a non-limiting example, the user may select any one of the various simulation positions of various specific organs input through the operating part 700, and the controller 500 may drive the driving part 300 and the auxiliary driving part 620 to implement the simulation position of the specific organ selected by the user in the training space (S1) by using the training target member 10.

In addition, the user may also finely adjust the position of the holder 200 by directly manipulating the driving part 300 through the operating part 700.

Through this, the user may finely change the position of the training target member 10 by manipulating the operating part 700 while the training target member 10 is implemented as the simulation position of a specific organ in the training space (S1), thereby allowing the user to more accurately change the training target member 10 to an intended simulation position.

Meanwhile, referring to FIG. 4, the endoscopic surgery training apparatus 1000 according to an embodiment of the present disclosure may further include a connector 103 provided on one side of the housing 100. The connector 103 is electrically connected to the holder support member 342 by a wire. The wire may be extended and arranged inside the housing 100 so as not to interfere with other components.

The holder 200 coupled to the holder support member 342 may be electrically connected to the connector 103. In this case, the contact portion of the holder support member 342 and the holder 200 may be electrically connected by having conductivity. In this case, the training target member 10 coupled to the holder 200 may be electrically connected to the connector 103.

The connector 103 is partially exposed to the outside of the housing 100 and is connected to a terminal of a cable that is electrically connected to a cauterizer. When the terminal of the cable is connected to the connector 103, the cauterizer may be electrically connected to the training target member 10 coupled to the holder 200 via the holder support member 342 and the holder 200.

Although the embodiments of the present disclosure have been described, the spirit of the present disclosure is not limited to the embodiments presented in the present specification, and those skilled in the art who understand the spirit of the present disclosure will be able to easily propose other embodiments by modifying, changing, deleting or adding components within the scope of the same spirit, but this will also be considered to fall within the spirit scope of the present disclosure.

### EXPLANATION OF REFERENCE NUMERALS

10: Training target member
20: Endoscopic device
1000: Endoscopic surgery training apparatus
100: Housing
103: Connector
110: First housing
120: Second housing
130: Door
140: Support plate
151: Space forming member
152: End plate
160: Partition wall
170: Coupling member
180: Rail
S1: Training space
S2: Storage space
S3: Placement space
200: Holder
210: Holder body
212: Mounting part
214: Leg part
215: Protrusion
220: Grasping member
230: First magnet member
240: Second magnet member
250: Fourth magnet member
300: Driving part
310: First linear driving part
311: First driving motor
312: Moving member
313: First guide rail
314: First slider
315: First rack gear
316: First pinion gear
320: First rotational driving part
321: Second driving motor
322: First rotational axis
323: First mounting member
323-1: First-1 mounting member
323-2: First-2 mounting member
324: First driving gear
325: First driven gear
330: Second rotational driving part
331: Third driving motor
332: Second rotational axis
333: Second mounting member
334: Second driving gear
335: Second driven gear
340: Second linear drive part
341: Fourth driving motor
342: Holder support member
342-1: First part
342-2: Second part
342a: Settling groove
343: Second guide rail
344: Second slider
345: Second rack gear
346: Second pinion gear
350: Cover member
360: Third magnet member
400: Channel forming part
410: Channel case
411: First side surface
412: Second side surface
413: Third side surface
414: First coupling groove
415: Second coupling groove
416: Third coupling groove
420: Path part
421: First path forming part
422: Second path forming part
423: Third path forming part
424: First entrance
425: Second entrance
430: Path maintenance member
431: First coupling port
432: Second coupling port
433: Connecting pipe
440: Exit forming member
500: Controller
600: Auxiliary support part
610: Auxiliary support
620: Auxiliary driving part
621: Fifth driving motor
622: Third slider
623: Third guide rail
624: Third rack gear
625: Third pinion gear
626: Guide roller
700: Operating part

1. An endoscopic surgery training apparatus, comprising:
a housing comprising a training space formed inside for training a surgery, and a support plate forming a bottom surface of the training space;
a holder configured to fix a training target member;
a driving part comprising a holder support member to which the holder is coupled, and configured to change a position of the holder coupled to the holder support member within the training space through at least one degree-of-freedom movement so as to change the training target member fixed to the holder to a specific simulation position of an organ for training within the training space;
a channel forming part formed in the housing so as to form an entry path of an endoscopic device entering the training space from the outside; and
a controller for controlling the driving part.

2. The endoscopic surgery training apparatus of claim 1, wherein the driving part changes the position of the holder coupled to the holder support member within the training space through a four-degree-of-freedom movement including a first linear movement, a first rotation, a second rotation and a second linear movement.

3. The endoscopic surgery training apparatus of claim 2, wherein the driving part comprises:
a first linear driving part configured to linearly move the holder support member along the X-axis direction;
a first rotational driving part configured to rotate the holder support member around a first rotation axis;
a second rotational driving part configured to rotate the holder support member around a second rotation axis; and
a second linear driving part configured to linearly move the holder support member in a direction perpendicular to the second rotation axis, and
wherein in the holder support member, a first part to which the holder is coupled is arranged so as to be parallel to the second rotation axis and to be spaced apart from the second rotation axis by a certain distance.

4. The endoscopic surgery training apparatus of claim 3, wherein when the holder support member is placed in an initial position by the driving part while the holder is coupled to the first part, the center of the holder is placed in a straight line with the first rotation axis, which is the central axis for a first rotation of the holder support member.

5. The endoscopic surgery training apparatus of claim 2, wherein the holder comprises a holder body comprising a mounting part that is detachably coupled to one side of the driving part and a plurality of leg parts that extend from the mounting part, and a plurality of gripping members that are detachably coupled to the plurality of leg parts so as to be able to grip the training target member.

6. The endoscopic surgery training apparatus of claim 5, wherein the holder comprises a plurality of first magnetic members that are provided on each of the plurality of leg parts, and a plurality of second magnetic members that are provided on each of the plurality of gripping members so as to correspond to the plurality of first magnetic members, and
wherein each of the plurality of gripping members is detachably coupled to the holder body through the first magnet member and second magnet member corresponding to each other.

7. The endoscopic surgery training apparatus of claim 2, wherein the driving part comprises:
a first linear driving part comprising a moving member that reciprocates linearly along the X-axis with respect to the support plate via a first driving motor;
a first rotational driving part comprising a second driving motor that is fixedly coupled to the moving member, a first rotation axis that rotates around the Z-axis that is perpendicular to the X-axis with respect to the moving member through a driving force of the second driving motor, and a first mounting member that is fixedly coupled to the first rotation axis;
a second rotational driving part comprising a third driving motor that is fixedly coupled to the first mounting member, a second rotation axis that rotates around an axis that is parallel to the XY plane and perpendicular to the Z axis with respect to the first mounting member through a driving force of the third driving motor, and a second mounting member that is fixedly coupled to the second rotation axis; and
a second linear driving part comprising a fourth driving motor that is fixedly coupled to the second mounting member, and provides a driving force for reciprocating a linear movement of the holder support member along a direction perpendicular to an axis that is parallel to the XY plane and perpendicular to the Z-axis with respect to the second mounting member.

8. The endoscopic surgery training apparatus of claim 7, wherein the endoscopic surgery training apparatus further comprises a third magnet member provided on the holder support member, and a fourth magnet member provided on the holder to correspond to the third magnet member, and
wherein the holder is detachably coupled to the holder support member via the third magnetic member and the fourth magnetic member.

9. The endoscopic surgery training apparatus of claim 7, wherein the first linear driving part further comprises:
a first guide rail fixed to the support plate;
a first slider movably coupled to the first guide rail so as to be able to move linearly in the X-axis direction along the first guide rail;
a first rack gear fixedly coupled to one side of the moving member so as to be parallel to the longitudinal direction of the first guide rail; and
a first pinion gear meshed with the first rack gear while being axially coupled to the first driving motor so as to be able to rotate by the first driving motor, and
wherein the moving member moves in a reciprocating linear manner along the X-axis with respect to the support plate through the first slider that is fixed to one side.

10. The endoscopic surgery training apparatus of claim 7, wherein the first rotational driving part further comprises a first driving gear that is axially coupled to the second driving motor, and a first driven gear that is axially coupled to the first rotation axis and meshes with the first drive gear, and
wherein the first mounting member rotates around the Z-axis, which is perpendicular to the X-axis, with respect to the moving member through a rotation of the first rotation axis.

11. The endoscopic surgery training apparatus of claim 7, wherein the second rotational driving part further comprises a second driving gear that is axially coupled to the third driving motor, and a second driven gear that is axially coupled to the second rotation axis and meshes with the second driving gear, and
wherein the second mounting member rotates around an axis perpendicular to the Z-axis with respect to the first mounting member through a rotation of the second rotation axis.

12. The endoscopic surgery training apparatus of claim 7, wherein the second linear driving part further comprises:
a second guide rail fixed to the second mounting member;
a second slider movably coupled to the second guide rail so as to be able to reciprocally move linearly along the second guide rail;
a second rack gear fixedly coupled to one side of the holder support member so as to be parallel to the longitudinal direction of the second guide rail; and
a second pinion gear meshed with the second rack gear while being axially coupled to the fourth driving motor so as to be able to rotate by the fourth driving motor, and
wherein the holder support member moves in a reciprocating linear manner along the second guide rail with respect to the second mounting member through the second slider that is fixed to one side.

13. The endoscopic surgery training apparatus of claim 1, wherein the controller drives the driving part such that the holder support member moves while the training target member is changed to a specific simulation position of an organ for training by driving the driving part.

14. The endoscopic surgery training apparatus of claim 1, wherein the channel forming part comprises:
a channel case coupled to the housing;
a path part formed to be recessed from one surface of the channel case so as to form an entry path for the endoscopic device; and
a path maintenance member formed in a hollow shape such that the endoscopic device passes through and detachably coupled to the path part,
wherein the path part comprises:
a first path forming part formed to be connected to the training space and have a certain length; and
a second path forming part and a third path forming part each extending from the first path forming part so as to branch in different directions from the other end of the first path forming part, and
wherein the path maintenance member is mounted on the path part so as to be positioned at the first path forming part and the second path forming part to form a first entry path, or is mounted on the path part so as to be positioned at the first path forming part and the third path forming part to form a second entry path.

15. The endoscopic surgery training apparatus of claim 14, wherein the path maintenance member comprises:
a hollow connecting pipe having a predetermined length;
a first coupling port provided at one end of the connecting pipe so as to be detachably coupled to one end of the first path forming part; and
a second coupling port provided at the other end of the connecting pipe so as to be detachably coupled to one end of the second path forming part or one end of the third path forming part.

16. The endoscopic surgery training apparatus of claim 3, wherein the endoscopic surgery training apparatus further comprises:
an auxiliary support part positioned at a bottom of the channel forming part so as to support a portion of the endoscopic device that has entered the training space through the channel forming part.

17. The endoscopic surgery training apparatus of claim 16, wherein the auxiliary support part comprises an auxiliary support configured to support a portion of an endoscopic device that has entered the training space, and an auxiliary driving part configured to change a position of the auxiliary support through a fifth driving motor within the training space, and
wherein the controller drives the auxiliary driving part to move the auxiliary support toward the training target member when the training target member changes to a preset specific simulation position.

18. The endoscopic surgery training apparatus of claim 17, wherein the auxiliary driving part further comprises:
a third slider fixed to a coupling member that is coupled to the support plate;
a third guide rail that is movably coupled to the third slider so as to be able to move linearly in the X-axis direction and has the auxiliary support fixedly coupled to one side;
a third rack gear formed along the X-axis direction on one side of the third guide rail; and
a third pinion gear that is axially coupled to the fifth driving motor so as to be able to rotate by the fifth driving motor and meshes with the third rack gear, and
wherein the auxiliary support moves toward the training target member through a linear movement of the third guide rail.

19. The endoscopic surgery training apparatus of claim 1, wherein the endoscopic surgery training apparatus further comprises:
an operating part for user operation,
wherein the operating part generates a user input signal to change the training target member to a specific simulation position of an organ.

20. The endoscopic surgery training apparatus of claim 1, wherein the endoscopic surgery training apparatus further comprises:
a connector provided on one side of the housing so as to be electrically connected to the holder support member, and a portion of which is exposed to the outside of the housing and is coupled to a terminal of a cable that is electrically connected to a cautery, and
wherein the holder that is coupled to the holder support member is electrically connected to the connector.
